(19) United States
(12) Patent Application Publication
(10) Pub. No.: US 2021/0379151 A1
(43) Pub. Date: Dec. 9, 2021

(54) USE OF VEGF AT MULTIPLE DOSES TO ENHANCE PERMEABILITY OF BLOOD BRAIN BARRIER

(71) Applicants: Ming-Che Shih, Los Angeles, CA (US); ACADEMIA SINICA, Taipei (TW)

(72) Inventors: Patrick C.H. Hsieh, Taipei (TW); David Lundy, Taipei (TW)

(21) Appl. No.: 17/282,514

(22) PCT Filed: Oct. 2, 2019
(86) PCT No.: PCT/US2019/054205

§ 371 Date: Apr. 2, 2021

Related U.S. Application Data
(60) Provisional application No. 62/740,840, filed on Oct. 3, 2018.

(51) Int. Cl. A61K 38/18; A61K 31/704; A61P 35/00; A61K 9/00

(52) U.S. Cl. CPC A61K 38/1866; A61K 9/0085; A61P 35/00; A61K 31/704

(57) ABSTRACT

A method of facilitating the delivery of an agent across the blood-brain barrier (BBB) of a subject, the method involving the use of a low dose of vascular endothelial growth factor (VEGF) polypeptide. In some embodiments, the VEGF polypeptide can be given to the subject before and after administration of the agent at multiple doses.

### CROSS REFERENCE TO RELATED APPLICATIONS

This application claims the benefit of the filing dates of U.S. Provisional Application No. 62/740,840, filed Oct. 3, 2018, the entire contents of which are incorporated by reference herein.

### BACKGROUND OF INVENTION

Glioblastoma Multiforme (GBM) is an aggressive primary cancer of the brain with a life expectancy of less than two years following diagnosis. Siegel et al., CA Cancer J Clin., 61, 212-236 (2011) and Bleeker et al., J. Neurooncol., 108 (1), 11-27 (2012). While great progress have been made in developing therapeutic agents for treating brain diseases such as GBM, it remains challenging to effectively deliver such therapeutic agents to disease sites in the brain due to the blood-brain barrier (BBB).

BBB is a highly selective two-way barrier system that separates systemic circulation from the brain parenchyma. The BBB preserves homeostasis of the brain by maintaining ion and neurotransmitter compartmentalisation, and controlling the transport of peptides, metabolites, cells and cytokines. As a result, the BBB prevents therapeutic drugs, for example, larger substances such as nanoparticles or liposomes, from passing into the brain following intravenous or oral administration. Azad et al., Neurosurg. Focus, 38 (7) (2015).

Direct injection of therapeutic agents to the brain was used to completely bypass the BBB. Xu et al., Biomaterials, 107, 44-60 (2016); Bago et al., Biomaterials, 90, 116-125 (2016); Fourniols et al., J. Control. Release, 210, 95-104 (2015); Chew et al., Adv. Healthc. Mater., 6 (2), 1600766 (2017); Wait et al., Neuro. Oncol., 17 (suppl 2), ii9-ii23 (2015); Baltes et al., J. Mater. Sci. Mater. Med., 21 (4), 1393-1402 (2010); and Debinski et al., Expert Rev Neurother, 9 (10), 1519-1527 (2013). However, these procedures are invasive and carry risks such as infection, haemorrhage, or damage to healthy brain tissue. Azad et al., 2015 and Debinski et al., 2013.

Accordingly, there exists a need to develop new approaches to facilitate delivery of therapeutic and diagnostic agents across the BBB for treating and/or diagnosing brain diseases, for example, GBM.

### SUMMARY OF INVENTION

The present disclosure is based, at least in part, on the unexpected discoveries that (i) VEGF165A creates a transient window (e.g., 45 minutes to 4 hours after systemic administration of the VEGF polypeptide), during which the blood brain barrier (BBB) has enhanced permeability, allowing for entry of therapeutic agents into the brain; and (ii) multiple low doses of VEGF showed enhanced effects in facilitating delivery of therapeutic agents, particularly large and/or water soluble molecules, to the brain. Surprisingly, administration of VEGF before and after the delivery of therapeutic agents, which may be encapsulated by a liposome or nanoparticle, further enhanced the efficacy of the therapeutic agents against brain tumors.

Accordingly, one aspect of the present disclosure features a method for delivering a therapeutic agent to the brain of a subject, the method comprising: (i) administering a first dose of a vascular endothelial growth factor (VEGF) polypeptide systemically to a subject in need thereof; (ii) administering to the subject systemically an effective amount of a therapeutic agent 15 minutes to 3 hours after step (i); and (iii) administering systemically a second dose of the VEGF polypeptide to the subject 2-24 hours after step (ii). In some embodiments, the second dose of the VEGF polypeptide in step (iii) is administered 2-8 hours after administration of the therapeutic agent in step (ii). For example, the second dose of the VEGF polypeptide in step (iii) can be administered 3-5 hours after administration of the therapeutic agent in step (ii).

In some embodiments, the method disclosed herein may further comprise (iv) administering a third dose of the VEGF polypeptide 2-24 hours after the second dose of the VEGF polypeptide in step (iii). In some examples, the third dose of the VEGF polypeptide can be administered 2-12 hours after the second dose of the VEGF polypeptide in step (iii). In some examples, the third dose of the VEGF polypeptide can be administered 3-5 hours after the second dose of the VEGF polypeptide in step (iii).

In some embodiments, the therapeutic agent can be administered to the subject about 45 minutes after the first dose of the VEGF polypeptide in step (i). Alternatively or in addition, the second dose of the VEGF polypeptide in step (iii) can be administered to the subject about 3 hours after administration of the therapeutic agent in step (ii). In some embodiments, the third dose of the VEGF polypeptide in step (iv) can be administered to the subject about 3 hours after administration of the second dose of the VEGF polypeptide in step (iii).

In any of the methods disclosed herein, the first dose, the second dose, and/or the third dose of the VEGF polypeptide is about 50-200 ng/kg. In some embodiments, the first dose, the second dose, and/or the third dose of the VEGF polypeptide is about 100-150 ng/kg.

The term “about” or “approximately” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

In another aspect, the present disclosure provides a method for facilitating delivery of a therapeutic agent across the BBB to the brain using a low dose of VEGF. Such a method may comprise: (i) administering a vascular endothelial growth factor (VEGF) polypeptide systemically to a subject in need thereof at a dose of 50-200 ng/kg (e.g., about 100-150 ng/kg); and (ii) administering to the subject a therapeutic agent 15 minutes to 3 hours after step (i). In some examples, the therapeutic agent is administered to the subject about 45 minutes after step (i).

The VEGF polypeptide for use in any of the methods disclosed herein can be a VEGF-A polypeptide. In some examples, the VEGF-A polypeptide can be human VEGF165A. In some embodiments, the VEGF polypeptide can be administered to the subject via an artery or a vein.

The therapeutic agent to be delivered by any of the methods disclosed herein can be a small molecule, a protein, or a nucleic acid. In some instances, the therapeutic agent is water soluble and/or has a molecular weight greater than 500 Dalton. In one example, the therapeutic agent is doxorubicin.

In some instances, the therapeutic agent can be is encapsulated by or attached to a liposome or a nanoparticle. In some examples, the liposome or the nanoparticle can be pegylated. The liposome or the nanoparticle disclosed herein may have a solid core diameter of about 20-500 nm, for example about 20-300 nm or about 20-200 nm. Such a solid core diameter may be determined by a routine method, for example, by transmission electron microscopy (TEM). See also Examples below.

In some instances, the therapeutic agent can be formulated in a pharmaceutical composition, which further comprises a pharmaceutically acceptable carrier. Alternatively, the therapeutic agent can be in free form.

The subject to be treated by any of the methods disclosed herein may be a human patient suspected of having, is at risk for, or a brain disease. Exemplary brain diseases include, but are not limited to, brain tumor (e.g., GBM), a brain stroke, a neuropsychiatric disorder, and a neurodegenerative disease.

Also provided herein are (i) a combination comprising a VEGF polypeptide as disclosed herein and a therapeutic agent as also described herein for use in treating a brain disease, wherein a low dose and/or multiple doses of the VEGF polypeptide facilitate delivery of the therapeutic agent to the brain, and (ii) uses of the just noted combination for treating a brain disorder or for manufacturing a medicament for use in treating the brain disorder.

The details of one or more embodiments of the invention are set forth in the description below. Other features or advantages of the present invention will be apparent from the following drawings and detailed description of several embodiments, and also from the appended claims.

### BRIEF DESCRIPTION OF DRAWINGS

FIGS. 1a-1g include diagrams showing that low-dose VEGF induced a transient increase in BBB permeability. FIG. 1a is a schematic diagram showing an exemplary experimental design. FIG. 1b is a diagram showing VEGF circulatory half-life following i.v. administration in mice, n=5 animals. FIG. 1c is a photo showing representative T1-weighted pre and post gadolinium (Gd)-enhanced MRI images of mouse brains, 45 minutes or 4 hours following VEGF or control administration. The regions of interest (ROI) of the cortex (blue), sinus (yellow) and noise (red) are shown. FIG. 1d includes charts showing quantification of signal to noise ratio in selected regions. Statistics analysis was performed using ANOVA with Tukey's HSD. Left panel: Cortex. Right panel: Sinus. FIG. 1e is a chart showing the biodistribution of Evans blue 45 minutes or 4 hours following VEGF pre-treatment. Statistics analysis was performed using ANOVA with Tukey's HSD. FIG. 1f includes photos depicting representative images showing isolectin (green) and Evans blue (red) in the cerebral cortex. Top left: control+Evans blue (Eb). Top right: pre-treatment with VEGF+Eb 45 minutes later. Bottom left: Cryolesion. Bottom right: blank control. Nuclei were stained with DAPI (blue). Cryolesion was used as a positive control. The scale bar is 100 μm. FIG. lg is a chart showing quantification of differently sized fluorescent PEG-modified polystyrene nanoparticles in the brain following control or VEGF pre-treatment. Statistics analysis of each size control vs. VEGF was performed by t-test. Error bars show standard error of the mean. Inset numbers indicate the number of animals. *p<0.05, **p<0.01, ***p<0.001 compared to control. ###p<0.001 compared to 4 hours. ns indicates not significant.

FIGS. 2a-2f include diagrams showing that VEGF enhanced delivery of selected anti-cancer drugs to the brain. FIG. 2a is a chart showing the quantification of Temozolomide (TMZ) in the brain of mice following pre-treatment with control (Ctrl+TMZ), VEGF (V+TMZ), or a ten-fold higher dose of VEGF (10×V+TMZ). TMZ was given at either 5 mg/kg or 20 mg/kg and circulated for one hour. Statistics analysis was performed by t-test vs. Ctrl+TMZ. FIG. 2b is a chart showing doxorubicin (dox) biodistribution 45 minutes following control or VEGF pre-treatment. Dox circulated for two hours before sample collection. Statistics analysis was performed by ANOVA with Tukey's HSD. FIG. 2c is a chart showing the percentage biodistribution of LipoDox, given 45 minutes following pre-treatment with control (Ctrl+LD) or VEGF (V+45 m LD). LipoDox was allowed to circulate for 4 hours before sample collection. Statistics analysis was performed by ANOVA with Tukey's HSD. FIG. 2d is chart showing organ concentrations of LipoDox normalized against the plasma concentration per mouse. Statistics analysis was performed by ANOVA with Tukey's HSD. FIG. 2e is a chart showing the effect of LipoDox (LD) and TMZ on DBTRG-05MG human glioblastoma cell viability, as determined by MTT assay. n=4. FIG. 2f is a chart showing LipoDox circulatory half-life following i.v. injection of 5 mg/kg by tail vein. n=5 animals. Error bars show standard error of the mean. Inset numbers indicate the number of animals tested. *p<0.05.

FIGS. 3a-3l include diagrams showing that VEGF enhanced drug delivery to the brain in a large animal model. FIG. 3a is a schematic diagram showing an exemplary experimental design of MRI studies in pigs. Top panel: exemplary experimental design of the study. Bottom panel: areas of the brain being analysed. TSE refers to turbo spin echo. FIG. 3b is a photo depicting a pig brain slice showing regions of interest. CTX, cerebral cortex; G, grey matter; W, white matter; HPF, hippocampal formation; TH, thalamus; STR—striatum (cerebral nuclei area); HY, hypothalamus; PIR, piriform area. Representative T1-weighted MRI images pre and post contrast in control and VEGF pre-treated pigs, and subtracted post:pre images showing a heatmap of the difference in normalised signal intensity scaled from 0 to 100. Quantification of SNR enhancement in selected brain regions. Results were compared using ANOVA with Tukey's HSD. FIG. 3c includes charts showing the average increase in SNR across all brain regions. Left panel: MRI post versus pre contrast SNR. Right panel: MRI SNR. Results were compared by unpaired t-test. FIG. 3d depicts a schematic diagram showing an exemplary experimental design to study drug biodistribution in pigs pre-treated with VEGF. FIG. 3e includes photos showing IVIS images showing nanoparticle fluorescence and pig brain accumulation. FIG. 3f is a chart showing HPLC-based quantification of nanoparticle systemic biodistribution. FIG. 3g is chart showing the nanoparticle distribution throughout brain areas. Data was analysed by ANOVA with Tukey's HSD. FIG. 3h is a chart showing the average brain retention of nanoparticles. Data was analysed by unpaired t-test. FIG. 3i is a chart showing a LipoDox systemic biodistribution. Data was analysed by ANOVA with Tukey's HSD. FIG. 3j is a chart showing a LipoDox brain distribution. Data was analysed by ANOVA with Tukey's HSD. FIG. 3k is a chart showing the average brain retention of LipoDox. Data was analysed by unpaired two-way t-test. FIG. 31 is a chart showing LipoDox concentration in CSF. Error bars show standard error of the mean. Inset numbers indicate the number of animals. *p<0.05, **p<0.01 compared to control. ns indicates not significant.

FIGS. 4a-4g include diagrams showing that VEGF affected multiple aspects of BBB permeability. FIG. 4a includes charts showing quantitative real-time PCR for key BBB genes 45 minutes and four hours following VEGF administration. n=4. Left panel: biochemical barriers. Middle panel: anatomical barriers. Right panel: others. Results from VEGF pre-treated animals were compared with results from control animals by Tukey's HSD. FIG. 4b includes photos showing the TEM imaging of brain blood vessels following VEGF administration. Panels from left to right: control, TEM imaging at 15 minutes, TEM imaging at 45 minutes, and TEM imaging at 4 hours. EC, endothelial cell; L, lumen; Er, erythrocyte; P, pericyte. Embedded scale bars are 1 μm. FIG. 4c includes photos showing the staining of pericyte marker PDGFRβ (red) and endothelial cell marker CD31 (green) in healthy brains and GBM xenografts. Panels from left to right: control, imaging at 15 minutes, imaging at 45 minutes, imaging at 4 hours, tumour control, and tumour with VEGF treatment. The average degree of pericyte coverage is shown in the upper right corner of each image. The scale bar is 100 μm. FIG. 4d includes photos showing staining of astrocyte marker GFAP (red) and endothelial cell marker CD31 (green) in healthy brains and GBM xenografts. Panels from left to right: control, imaging at 15 minutes, imaging at 45 minutes, imaging at 4 hours, tumour control, and tumour with VEGF treatment. The scale bar is 100 um. FIG. 4e include photos showing immunofluorescence images of tight junction protein claudin 5 (red, middle row) and endothelial cell marker CD31 (green, top row) in healthy brains and GBM xenografts. Separate channels and a merged image are shown. The bottom row shows merged image of the top and middle rows. The colocalisation coefficient is shown in the upper right of each image. The scale bar is 40 μm. FIG. 4f is a chart showing average pericyte coverage. FIG. 4g is a chart showing average claudin 5 colocalisation. Error bars show standard error of the mean. Inset numbers indicate number of animals. *p<0.05, **p<0.01, ***p<0.001, ****p<0.0001 compared to control. ns indicates not significant.

FIGS. 5a-5k include diagrams showing LipoDox in combination with VEGF pre-treatment extended animal survival in a mouse model of glioblastoma. FIG. 5a is a schematic diagram of an exemplary experimental design showing time course and explanation of VEGF (V) and multiple VEGF (MV) treatment courses. FIG. 5b includes a chart showing the quantification of intratumoural LipoDox concentration in tumour-bearing mice. GBM xenografts and the contralateral region from the same animal were analysed. Statistics analysis was performed using t-test. FIG. 5c includes a chart showing a Kaplan-Meier survival curve. Pairs of curves are compared by Log-rank (Mantel-Cox) test. FIG. 5d includes photos and corresponding charts showing a weekly summary of tumour luminescence in each treatment group. The number of animals at each time point is inset and representative IVIS images are shown. The data was analysed by ANOVA with Tukey's HSD. FIG. 5e includes diagrams showing a tumour volume analysis, as determined by MRI at day 45. Left panel: charts showing tumour volumes. Right panel: photos showing tumour imaging. Representative 1 mm thick slices (slices 12, 13 and 14) are shown, with the tumour area marked by a white boundary. Data was analysed by unpaired t-test. FIG. 5f includes diagrams showing a Ki67 analysis of tumour sections from mice which died between days 60 and 70. Representative images show Ki67 (green) and DAPI (blue). Scale bar 100 μm. Data was analysed by ANOVA with Tukey's HSD. FIG. 5g includes a chart showing intratumoural cell density determined by DAPI staining. Results were analysed by ANOVA with Tukey's HSD. FIG. 5h includes a chart showing tumour blood vessel density per 400× magnification field, as determined by isolectin staining. For sham mice, the injected region was imaged. Data was analysed by ANOVA with Tukey's HSD. FIG. 5i is a chart showing quantification of Ibal positive cell content in brain tumour. Data was analysed by ANOVA with Tukey's HSD. FIG. 5j is a chart showing quantification of intratumoural oedema, as determined by H&E staining. For sham, an equal-sized area of normal brain was analysed. Data was analysed by ANOVA with Tukey's HSD. FIG. 5k is a chart showing quantification of intratumoural haemorrhage, as determined by H&E staining. For sham, an equal-sized area of normal brain was analysed. Data was analysed by ANOVA with Tukey's HSD. Error bars show standard error of the mean. Inset numbers indicate the number of animals analysed. *p<0.05, **p<0.01, ***p<0.001. ns indicates not significant.

FIGS. 6a-6d include diagrams showing that lose dose intravenous administration of VEGF did not raise safety concerns. FIG. 6a is a chart showing the quantification of plasma 51000 concentration in mice. Lipopolysaccharide (LPS) to induce BBB disruption was used as positive control. Brain lysate was used as a second positive control. Before and after samples were analysed by paired two-way t-test. n>4 per group. FIG. 6b is a chart showing mouse systolic and diastolic blood pressure measured every 30 minutes for four hours following VEGF or a ten-fold dose. The first sample (0 minutes) was taken immediately prior to VEGF administration. FIG. 6c is a chart showing the changes in pig blood systolic and diastolic blood pressure after VEGF administration. Data was analysed by paired t-test. FIG. 6d includes charts showing gene expression of key neuroinflammation markers 45 minutes and four hours following VEGF administration. n>5. Top row from left to right: TNF, IL1b, and IL6. Bottom row from left to right: CCL2, CXCL2, and GFAP. Cryolesion injury (cryo) and LPS were used to induce neuroinflammation. Each sample was normalised against Gapdh. Each group analysed vs. PBS, and 4 hrs vs. 24 hrs by two-way ANOVA with Tukey's HSD. Average threshold cycle numbers (C_{T}) for the PBS group are shown for reference. Error bars show standard error of the mean. Inset numbers indicate the number of animals. *p<0.05, **p<0.01, ***p<0.001, ****p<0.0001 compared to control. #p<0.05, ##p<0.01, ###p<0.001 compared to 4 hours. ns indicates not significant.

FIG. 7 includes diagrams showing the penetration of IgG antibody into the brain and penetration of anti-nrCAM IgG primary antibody into brain tissue. Primary antibody was injected intravenously, 45 minutes following control or VEGF, then the animal was perfusion fixed, the brain was frozen sectioned, and stained with fluorescent secondary antibody. For the I.C. Ab sample, anti-nrCAM was directly injected intracranially. A section stained by conventional methods is also shown for reference.

FIGS. 8a-8c include charts showing standard curves for Evans blue, Temozolomide (TMZ) and doxorubicin as determined by HPLC. FIG. 8a: standard curve for Evans blue. FIG. 8b: standard curve for TMZ. FIG. 8c: standard curve for doxorubicin (HPLC).

FIGS. 9a-9d include charts showing LipoDox and nanoparticle HPLC quantification results. FIG. 9a: standard curve of low concentration (<1.0 μg/ml) LipoDox. FIG. 9b: standard curve of high concentration (<300.0 μ/ml) LipoDox. FIG. 9c: LipoDox recovery from brain tissue. Dotted lines indicate 90% and 110% margins. FIG. 9d: standard curves of HPLC-based nanoparticle quantification, with and without the presence of LipoDox. Left panel: peak area under different concentrations of the agent as indicated. Right panel: retention time chart indicating that presence of LipoDox does not affect nanoparticle dye retention time.

FIG. 10 is a chart showing the effect of VEGF on DBTRG cell viability. DBTRG cells were cultured with VEGF up to a concentration of 100 ng/ml.

FIGS. 11a-11b include diagrams showing expression of claudin 5 and P-glycoprotein in response to VEGF treatment. FIG. 11a shows results from a western blot analysis of whole mouse brain Claudin 5 following VEGF treatment. Left panel: a chart quantifying Claudin5 relative expression percentage. Right panel: a photo showing expression of Glaudin5 at various time points as indicated. FIG. 11b include photos shows P-glycoprotein staining following VEGF treatment at various time points as indicated. Scale bar=100 μm.

FIGS. 12A-12B include charts showing biodistribution of doxorubicin or LipoDox following VEGF treatment. FIG. 12A: a chart showing doxorubicin biodistribution following VEGF pre-treatment in mice. FIG. 12B is a chart showing LipoDox biodistribution following multiple doses of VEGF treatment in mice.

FIGS. 13a-13c include diagrams showing various aspect of the GBM mouse model used in this study. FIG. 13a includes diagrams showing luciferase expression in engineered DBTRG-05MG human glioblastoma cell line. Left panel: a chart showing the level of luciferase expression in the DBTRG cells. Right panel: a photo showing luciferase signal in the DBTRG cells. FIG. 13b is a photo showing an example BALB/c NU mouse receiving intracranial injection. FIG. 13c is a photo showing a typical tumour morphology in right hemisphere after 65 days.

FIGS. 14a-14b include diagrams showing the effect of sham injections on drug retention. Intratumoral Lipodox following V+LD treatment. FIG. 14a is a chart showing LipoDox concentration at the sham injection site or contralateral side in mice. FIG. 14b is chart showing intratumoural LipoDox concentration following a single dose of VEGF followed by LipoDox.

FIGS. 15a-15e include diagrams showing characteristics of mice having brain tumor and treated with LD either alone or with VEGF pre-treatment. FIG. 15a is a chart showing correlation of tumor luminescence determined by IVIS vs. confirmed tumor size by MRI. FIG. 15b is a chart showing mouse body weight throughout survival experiment. FIG. 15c includes photos showing Iba1 staining of tumors from mice in treatment groups. The corresponding normal brain region was imaged in the sham mice. Scale bar=100 μm. FIG. 15d is a photo showing an example H&E image showing tumor with areas of edema and hemorrhage. FIG. 15e is a chart showing correlation of IVIS-based luminescence measurement as related to MRI-determined tumour volumes.

FIGS. 16a-16d include diagrams showing characteristics of the PDAC model. FIG. 16a includes a chart (left) and a photo (right) showing IVIS conforming luciferase expression of AsPC1 cells. FIG. 16b is a photo showing IVIS showing pancreatic tumor establishment in mice. FIG. 16c includes exemplary photos showing an normal pancreas and PDAC xenograft pancreas. FIG. 16d is a chart showing a quantification of LipoDox in PDAC tumors or sham-operated pancreas.

FIGS. 17a-17c include diagrams showing characteristics of the subcutaneous GBM mouse model. FIG. 17a is a photo showing a representative IVIS image of subcutaneous tumor growth. FIG. 17b is a photo showing a representative tumor after 60 days. FIG. 17c is a chart showing the intratumoral LipoDox concentration following control or VEGF pre-treatment.

FIGS. 18a-18b include charts showing supplementary 45 minute, 4 hour and 24 hour inflammation gene expression. FIG. 18a includes charts shows expression of Fn1 (left) and Il1a (right) following treatment groups. Cryolesion (cryo) and lipopolysaccharide (LPS) were used to induce neuroinflammation as positive controls. FIG. 18b includes charts showing gene expression 45 minutes following VEGF administration. Top row from left to right: IL1b at 45 minutes, TNFa at 45 minutes, and IL6 at 45 minutes. Bottom row from left to right: CCL2 at 45 minutes, CXCL1 at 45 minutes, and GFAP at 45 minutes.

FIG. 19 includes charts showing mouse serum blood chemistry. Top row from left to right: ALT/GPT (alanine Aminotransferase); CPK (creatinine kinase); and LDH (lactate dehydrogenase). Bottom row from left to right: ALP (alkaline phosphatase); BUN (blood urea nitrogen; and CK-MB (creatinine kinase MB).

### DETAILED DESCRIPTION OF INVENTION

A number of approaches have been tried to overcome the challenges associated with drug delivery across the BBB, including disruption of the BBB, permeating the BBB, bypassing the BBB, or a combination thereof. Osmotic treatments can disrupt the barrier, and many attempts have been made to utilize endogenous carrier proteins for drug uptake and delivery. The BBB may be avoided entirely by direct injection of drugs into cerebrospinal fluid or directly into the brain. However, these methods present their own challenges such as ion imbalances, leaking neurotransmitters and chemokine release into circulation. Obermeier et al., Nat Med 19(12): 1584-1596; 2013.

Provided herein is an advantageous method involving systemic administration of a low dose of vascular endothelial growth factor (VEGF) before administration of a therapeutic agent or multiple low doses of the VEGF before and after administration of the therapeutic agent to enhance permeability of the BBB, thereby improving brain intake of the therapeutic agent. This advantageous method is based on the unexpected discoveries reported herein showing the effects of VEGF on BBB permeability. Some examples are provided below.

The present studies show that a low dose of intravenous injection of VEGF created a transient window (about 45 minutes to 4 hours), during which the permeability of the BBB is enhanced and the BBB restores its integrity after this window. Further, the present studies show that multiple doses of VEGF, e.g., one dose before administration of a therapeutic agent, and one or more doses after administration of the therapeutic agent, are more effective in facilitating therapeutic agents such as nanoparticle- or liposome-based agents across the BBB, thereby enhancing the intended therapeutic efficacy, for example, greatly extending survival in a mouse model of human glioblastoma.

Further, similar results were observed in both a small animal model (a mouse model) and in a large animal model (a pig model), indicating that the selected doses for VEGF would be expected to be effective in human therapy. In particular, the results reported herein show that VEGF-pretreatment enhanced entry of therapeutic agents (e.g., LipoDox as an example) into brain tumour regions at a much higher level than entry of the therapeutic agents into normal brain regions as observed in a mouse model.

Moreover, the low dose of VEGF, unexpectedly, was not found to increase tumour vasculogenesis, induce hypotension, or cause any obvious adverse effects. A ten-fold higher dose of the VEGF also did not disrupt compartmentalisation of the brain; nor did it induce significant hypotension.

Inhibition of VEGF signalling is an important therapeutic target in cancer treatment. Kim et al., Nature, 362 (6423), 841-844 (1993). Therefore, administering exogenous VEGF to cancer patients is surprising and appears counter-intuitive. VEGF has previously been shown to be a potent inducer of inflammation and can cause hypertension. Surprisingly, the results of the present studies showed that VEGF only induced very mild inflammation. Hypotension was not detected in a 3 hour period following VEGF administration. Since VEGF was found to induce neuroinflammation, it is expected that multiple, low doses of VEGF can enhance therapeutic efficacy and minimize side effects.

### Use of VEGF to Facilitate Delivery of Therapeutic/Diagnostic Agents Across the BBB

One aspect of the present disclosure features methods of treating brain diseases that involve the co-use of a VEGF polypeptide at a low dose and/or multiple doses and an agent (e.g., a diagnostic agent or a therapeutic agent). The VEGF polypeptide can be systemically administered to a subject in need of the treatment at a low dose, followed by administration of the agent within a suitable time window after administration of the VEGF polypeptide. Optionally, the VEGF polypeptide may be given to the subject one or more times after administration of the agent within a suitable timeframe.

(i) VEGF

Vascular endothelial growth factor (VEGF) is a signal protein produced by cells that stimulates vasculogenesis and angiogenesis. It is a growth factor that belongs to the platelet-derived growth factor sub-family. The normal function of VEGF is to create new blood vessels during embryonic development, new blood vessels after injury, muscle following exercise, and new vessels (collateral circulation) to bypass blocked vessels. Vascular endothelial growth factor (VEGF) is a soluble homodimeric protein responsible for the normal formation of new blood vessels, as well as promoting cell growth and survival.

Five forms of VEGF are found in humans, with VEGF165A being the predominant form found in normal cells and tissues. Ferrara et al., Nat Med, 9 (6), 669-676 (2003). VEGF acts through binding to the VEGFR-1 receptor or the VEGFR-2 receptor presented on endothelial cells, and has been long-known to affect vascular permeability. Senger et al., Science, 219 (4587), 983-985 (1983); Connolly et al., Regulation of Vascular Function by Vascular Permeability Factor. In Vascular Endothelium: Physiological Basis of Clinical Problems; Catravas, J. D., Callow, A. D., Gillis, C. N., Ryan, U. S., Eds.; Springer U S: Boston, Mass., 1991; pp 69-76; and Lee et al., J. R. Soc. Interface, 8 (55), 153-170 (2011). Given its role in angiogenesis, VEGF has undergone human clinical trials for use in ischaemic diseases, where it was found to be well tolerated, although not particularly efficacious. Henry et al., Circulation, 107 (10), 1359-1365 (2003).

VEGF is also known to play a role in pathophysiological angiogenesis, and therapies focusing on reducing free circulating VEGF (bevacizumab) or interfering with VEGFR activity (cediranib) have been successfully used to slow tumour progression by reducing nutrient delivery and interfering with cell survival pathways. Khasraw et al., In Cochrane Database of Systematic Reviews; 2014; Kim et al., 1993; Weis et al., Nature, 437 (7058), 497-504 (2005); and Lu-Emerson et al., J. Clin. Oncol., 33 (10) (2015). These drugs may also normalise tumour vasculature, resulting in more effective drug delivery to tumours. Jain et al., Science, 307:58-62 (2005).

VEGF of any of the five families noted herein can be used for the method disclosed herein. The VEGF can be from a suitable origin, e.g., human, monkey, mouse, rat, pig, dog, and cat. In some embodiments, the VEGF molecule used in the methods described herein is a VEGF-A molecule, such as the VEGF-A₁₆₅ isoform. The amino acid sequence of the human VEGF-A₁₆₅ is:

(SEQ ID NO: 1)
APMAEGGGQNHHEVVKFMDVYQRSYCHPIETLVDIFQEYPDEIEYIFKP
SCVPLMRCGGCCNDEGLECVPTEESNITMQIMRIKPHQGQHIGEMSFLQ
HNKCECRPKKDRARQENPCGPCSERRKHLFVQDPQTCKCSCKNTDSRCK
ARQLELNERTCRCDKPRR.

In some instances, the VEGF molecule used in the methods described herein is a wild-type VEGF. In other instances, it can be a modified variant, which preserves the same or similar bioactivity as the wild-type counterpart.

Such a modified variant may share a sequence identity of at least 85% (e.g., 90%, 95%, 97%, 99%, or above) relative to the wild-type counterpart. The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the modified variant consists of one or more conservative amino acid residue substitutions as compared with the wild-type counterpart. The skilled artisan will realize that conservative amino acid substitutions may be made in a VEGF molecule to provide functionally equivalent variants, i.e., the variants retain the functional capabilities of the particular VEGF. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Conservative amino-acid substitutions in the amino acid sequence of a VEGF to produce functionally equivalent variants typically are made by alteration of a nucleic acid encoding the mutant. Such substitutions can be made by a variety of methods known to one of ordinary skill in the art. For example, amino acid substitutions may be made by PCR-directed mutation, site-directed mutagenesis according to the method of Kunkel (Kunkel, PNAS 82: 488-492, 1985), or by chemical synthesis of a nucleic acid molecule encoding a VEGF variant.

Any of the VEGF molecules for use in the methods described herein may be prepared by conventional methods. For example, the molecule can be isolated from a suitable natural source following the routine protein purification procedures. Alternatively, it can be produced in a suitable host cell via the conventional recombinant technology.

Besides VEGF, other growth factors such as IGF-I and IGF-II, may also be used in the methods described herein.

(ii) Therapeutic and Diagnostic Agents

The method disclosed herein aims at facilitating delivery of an agent across the BBB to the brain, wherein the agent can exert tis intended activity. In some instances, the agent can be a therapeutic agent for treating a brain disorder, for example, a brain tumor. In other instances, the agent can be a diagnostic agent, e.g., an imaging agent, for diagnosing a brain condition.

In some embodiments, the therapeutic agent or diagnostic agent disclosed herein may have a half-life of at least 1 hour, at least 5 hours, at least 10 hours, at least 15 hours, at least 20 hours, at least 24 hours, at least 36, hours, at least 48, hours, at least 72 hours, at least 25 hours, at least 30 hours, at least 35 hours, at least 40 hours, at least 45 hours, at least 50 hours, at least 55 hours, at least 60 hours, at least 65 hours, at least 70 hours, at least 75 hours, at least 80 hours, at least 85 hours, at least 90 hours, at least 95 hours, or at least 100 hours. For example, the therapeutic agent may have a half-life of at least 40 hours. In some instances, a long half-life may be a half-life of at least 24 hours, at least 30 hours, at least 35 hours, at least 36 hours, at least 40 hours, at least 44 hours, at least 45 hours, at least 50 hours, 50 hours, at least 55 hours, at least 60 hours, at least 65 hours, at least 70 hours, at least 75 hours, at least 80 hours, at least 85 hours, at least 90 hours, at least 95 hours, at least 100 hours, at least 1 week, at least 2 weeks, at least 3 weeks, at least 4 weeks, at least 5 weeks, at least 6 weeks, at least 7 weeks, at least 8 weeks, at least 5 months, or at least one year.

The therapeutic agent disclosed herein can be any molecule that possesses one or more therapeutic effects. Such a molecule can be a small molecule, a protein (e.g., an antibody), a nucleic acid (e.g., an antisense oligonucleotide, an aptamer, or an interfering RNA), a lipid, or a sugar. In some instances, the therapeutic agent can be a water soluble compound. Alternatively or in addition, the therapeutic agent can be a small molecule (e.g., having a molecule weight no greater than 5000 Dalton) have a relatively large size, for example, having a molecule weight of greater than 500 Dalton, for example, greater than 1 kDa, greater than 2 kDa, greater than 3 kDa, or greater than 4 dKa.

In some embodiments, the therapeutic agent can be in free form. Alternatively, the therapeutic agent can be conjugated to a carrier, covalently or non-covalently. In some embodiments, the therapeutic agent may be embedded in, encapsulated by, or attached to a liposome or a nanoparticle.

In some instances, the agent (e.g., a therapeutic agent or a diagnostic agent, optionally the VEGF polypeptide) can be embedded in, encapsulated by, or attached to a liposome. For example, the liposomes may have the active agents inside the liposome or the active agents may be embedded on the surface of the liposome. As an example, the therapeutic agents of the present disclosure may be encapsulated by or embedded in a liposome. As a non-limiting example, the therapeutic agent may be liposomal doxorubicin (LipoDox). See, e.g., U.S. Patent Publication Number US 5,213,804. Liposomes comprising an active agent (e.g., the VEGF polypeptide, the diagnostic agent, the therapeutic agent, or any combination thereof) can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

A liposome may be neutrally charged. The charge of a liposome may be determined using a zeta potential measurement. See, e.g., Clogston and Patri, Methods Mol Biol. 2011; 697:63-70. For example, a neutrally charged liposome may comprise a zeta potential between −10 mV and +10 mV (e.g., between −5 mV and 0 mV, between −3 mV and 0 mV, between −2 mV and 0 mV, between 0 and 5 mV, between −2 mV and 2 mV, or between −10 mV and −5 mV, between 5 mV and 10 mV).

Alternatively, the active agents (e.g., a therapeutic agent, a diagnostic agent, or optionally the VEGF polypeptide) may also be entrapped in microcapsules to form nanoparticles. Such nanoparticles may be prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

Any of the liposomes or nanoparticles disclosed herein may have a suitable size, for example, a suitable solid core diameter or a suitable hydrodynamic diameter, which can be determined by conventional methods, for example, transmission electron microscopy and Malvern Zetasizer, respectively. In certain instances, the liposomes or nanoparticles may comprise polyethylene glycol (PEG).

In some instances, a suitable solid core diameter of the liposomes and nanoparticles disclosed herein may range from about 20-500 nm, e.g., about 20-400 nm, about 20-300 nm, about 20-250 nm, about 20-200 nm, about 20-150 nm, about 20-100 nm, about 50-300 nm, about 50-200 nm, or about 100-300 nm. Alternatively or in addition, a suitable hydrodynamic diameter of the liposomes and nanoparticles disclosed herein may range from 30-550 nm, e.g., about 30-500 nm, about 30-450 nm, about 30-350 nm, about 30-300 nm, about 30-250 nm, about 50-250 nm, or about 150-350 nm. In some embodiments, the hydrodynamic diameter of a liposome may be less than 100 nm (e.g., between 10 nm and 100 nm, between 20 nm and 100 nm, between 30 nm and 100 nm, between 40 nm and 100 nm, between 50 nm and 100 nm, between 60 nm and 100 nm, between 70 nm and 100 nm, between 80 nm and 100 nm, between 90 and 100 nm, between 91 nm and 100 nm, between 90 and 95 nm, between 95 and 100 nm, between 92 nm and 100 nm, between 93 nm and 100 nm, between 94 nm and 100 nm, between 96 and 100 nm, between 97 nm and 100 nm, between 98 nm and 100 nm, or between 99 nm and 100 nm. The hydrodynamic diameter of a liposome may be measured using any suitable technique, including dynamic light scattering. See, e.g., Kaszuba et al., J Nanopart Res (2008) 10: 823 and the examples below.

The therapeutic agent may be an anti-cancer agent, for example, an agent for treating a brain tumor such as glioblastoma. Non-limiting examples of anti-cancer agents include topoisomerase inhibitors (e.g., camptothecin, irinotecan, topotecan, etoposide, doxorubicin, teniposide, novobiocin, merbarone, and aclarubicin); anti-metabolites (e.g., fluoropymidine, deoxynucleoside analogue, thiopurine, methotrexate, and pemetrexed); alkylating agents (e.g., cisplatin, carboplatin, oxaliplatin, mechlorethamine, cyclophosphamide, melphalan, chlorambucil, ifosfamide, busulfan, N-nitroso-N-methylurea (MNU), carmustine, lomustine, semustine, fotemustine, streptozotocin, dacarbazine, mitozolomide, temozolomide, thiotepa, mytomycin, and diaziquone); a cytotoxicity antibiotic (e.g., actinomycin, bleomycin, plicamycin, mitomycin, doxorubicin, daunorubicin, epirubicin, idarubicin, piraubicin, alcarubicin, and mitoxantrone), or biologics (e.g., a therapeutic antibody such as Bevacizumab, Cetuximab, Pemtumomab, oregovomab, minretumomab, Etaracizumab, Volociximab, Cetuximab, panitumumab, nimotuzumab, Trastuzumab, pertuzumab, AVE1642, IMC-Al2, MK-0646, R1507, CP 751871, Mapatumumab, KB004 or IIIA4).

In certain instances, the therapeutic agent (e.g., anti-cancer agent) is encapsulated by or embedded in a liposome. A non-limiting example of a therapeutic agent encapsulated by a liposome is liposomal doxorubicin. Doxorubicin is a chemical compound that intercalates in DNA and has been implicated in inhibiting topoisomerase II. As a non-limiting example, doxorubicin may comprise formula I shown below.

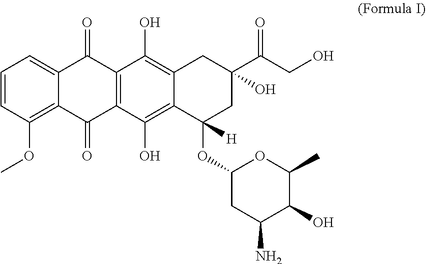 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1]

It should be appreciated that doxorubicin derivatives and pharmaceutically acceptable salts thereof are also encompassed by the present disclosure. For example, doxorubicin may be doxorubicin hydrochloride.

In some instances, one or more positions in Formula I may be modified (e.g., through substitution or addition of a functional group). Non-limiting examples of functional groups include hydrocarbons chains (e.g., substituted or unsubstituted alkyl, alkenyl, or alkynyl groups), benzene rings, amine groups, alcohols, ethers, alkyl halides, thiols, aldehydes, ketones, esters, carboxylic acids, and amides. Accordingly, the term “doxorubicin” as used herein encompasses any of these modified variants of Formula I.

The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N.Y., 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

(iii) Pharmaceutical Compositions

Any of the active agents for use in the methods described herein (e.g., the VEGF, the diagnostic agent, and/or the therapeutic agent) can be mixed with a pharmaceutically acceptable carrier (excipient), including buffer, to form a pharmaceutical composition for use in any of the methods disclosed herein. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

The pharmaceutical compositions to be used for in vivo administration must be sterile. This is readily accomplished by, for example, filtration through sterile filtration membranes. Therapeutic compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 .im, particularly 0.1 and 0.5 .im, and have a pH in the range of 5.5 to 8.0.

The emulsion compositions can be those prepared by mixing a VEGF or a therapeutic agent with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

In one example, one or more of the active agents may be formulated into liquid pharmaceutical compositions, which are sterile solutions, or suspensions that can be administered by, for example, intravenous, intramuscular, subcutaneous, or intraperitoneal injection. Suitable diluents or solvent for manufacturing sterile injectable solution or suspension include, but are not limited to, 1,3-butanediol, mannitol, water, Ringer's solution, and isotonic sodium chloride solution. Fatty acids, such as oleic acid and its glyceride derivatives are also useful for preparing injectables, as are natural pharmaceutically-acceptable oils, such as olive oil or castor oil. These oil solutions or suspensions may also contain alcohol diluent or carboxymethyl cellulose or similar dispersing agents. Other commonly used surfactants such as Tweens or Spans or other similar emulsifying agents or bioavailability enhancers that are commonly used in manufacturing pharmaceutically acceptable dosage forms can also be used for the purpose of formulation.

(iv) Facilitating Brain Delivery of Therapeutic or Diagnostic Agents

Any of the VEGF polypeptides disclosed herein, for example, VEGF-A such as VEGF165A (as well as other growth factors), can be used in combination with any of the agents also disclosed herein (e.g., a therapeutic agent or a diagnostic agent) to enhance delivery of the agent across the BBB to the brain. A low dose of the VEGF polypeptide can be given a subject in need of the treatment first and within a suitable window after administration of the VEGF, a suitable dose of the agent can be administered to the subject via a suitable route. In some instances, one or more additional doses of the VEGF polypeptide can be given to the subject within a suitable time period after the administration of the agent. Two consecutive VEGF doses may be given to the subject systematically within a suitable time period, e.g., about 2-24 hours apart.

Any of the therapeutic or diagnostic agents disclosed herein may be used in combination with VEGF to facilitate brain delivery. In some instances, the therapeutic or diagnostic agent may be embedded in or encapsulated by a liposome or a nanoparticle.

To perform the methods described herein, a pharmaceutical composition comprising a suitable amount of a VEGF polypeptide (e.g., human VEGF-A165) can be administered to a subject in need of the treatment (e.g., as those described herein) first via a suitable route, for example, intravenous injection, intra-arterial injection, or subcutaneous injection. After a suitable period of time, a pharmaceutical composition comprising an effective amount of a therapeutic or diagnostic agent can be given to the same subject via a suitable route.

The term “administered”, “administering” or “administration” are used interchangeably herein to refer a mode of delivery, including, without limitation, intravenously, intramuscularly, intraperitoneally, intraarterially, intracranially, or subcutaneously administering an agent (e.g., a compound or a composition) of the present invention. In one embodiment of the present disclosure, the growth factor (e.g., VEGF) the therapeutic agent or the diagnostic agent such as a contrast agent for imaging is administered to the subject by direct intravenously or intracranially injection. Systemic administration is a route of administration of an agent into the circulatory system so that the entire body is affected. Administration can take place via enteral administration (absorption of the drug through the gastrointestinal tract) or parenteral administration (injection, infusion, or implantation).

The VEGF polypeptide (as well as another growth factor as disclosed herein) and the therapeutic/diagnostic agent, may be administered to a suitable subject (e.g., a mammal, such as a human) by any route that may effectively transports the VEGF and/or the therapeutic/diagnostic agent to the appropriate or desired site of action. Exemplary administration routes include, but are not limited to, oral, nasal, pulmonary, transdermal, such as passive or iontophoretic delivery, or parenteral, e.g., rectal, depot, subcutaneous, intravenous, intramuscular, intranasal, intra-peritoneal, intra-arterial, intra-cranial, intra-cerebella, subcutaneous, ophthalmic solution or an ointment.

In some embodiments, the VEGF polypeptide (as well as other growth factors) and/or the therapeutic/diagnostic agent can be administered via a conventional systemic route, for example, intravenous injection or subcutaneous injection. Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble agents such as VEGF or the therapeutic/diagnostic agent can be administered by the drip method, whereby a pharmaceutical formulation containing the agent and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the agent, can be dissolved and administered in a pharmaceutical excipient such as Water-for-Injection, 0.9% saline, or 5% glucose solution.

In some embodiments, the VEGF polypeptide may be administered to a subject at a low dose. For example, the VEGF is administered to a subject (e.g., a human subject) in the amount of about 10 ng/kg to 500 ng/kg, for example, about 20-250 ng/kg, about 50-200 ng/kg, or about 100-150 ng/kg. The selected dose of VEGF should be high enough to enhance the permeability of BBB, but insufficient to disrupt the integral structure of BBB that inevitably leads to subsequent damage to the brain (e.g., edema). Accordingly, VEGF is preferably to be administered to the subject (e.g., a human subject) in the amount of about 10 ng/kg to 500 ng/kg, such as about 20 ng/kg, 50 ng/kg, 80 ng/kg, 100 ng/kg, 120 ng/kg, 150 ng/kg, 180 ng/kg, 200 ng/kg, or 250 ng/kg. For subjects who are sensitive to VEGF, the dose of VEGF may be reduced, for example, to less than 10 ng/kg (e.g., about 1-5 ng/kg or lower). Alternatively, for subjects who are tolerable to VEGF, the dose of VEGF may be increased, for example, to greater than 500 ng/kg (e.g., about 500 ng/kg to 5 μg/kg such as 800 ng/kg, 1 μg/kg, 2 μg/kg, 3 μg/kg, 4 μg/kg, or 5 μg/kg).

An effective amount of the therapeutic agent or the diagnostic agent is co-used with the VEGF polypeptide (or another growth factor) for treating or diagnosing a brain disorder in a subject. The term “an effective amount” as used herein refers to an amount effective, at dosages, and for periods of time necessary, to achieve the desired result with respect to the treatment of a disease. For example, in the treatment of a cancer, an agent (i.e., a compound or a composition) which decrease, prevents, delays or suppresses or arrests any symptoms of the cancer would be effective. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two or more doses in a suitable form to be administered at one, two or more times throughout a designated time period.

It will be appreciated that the dosage of the VEGF (or other growth factors) and/or the therapeutic agent of the present disclosure will vary from patient to patient not only for the particular growth factor or therapeutic agent selected, the route of administration, and the ability of the growth factor or the therapeutic agent to elicit a desired response in the patient, but also factors such as disease state or severity of the condition to be alleviated, age, sex, weight of the patient, the state of being of the patient, and the severity of the pathological condition being treated, concurrent medication or special diets then being followed by the patient, and other factors which those skilled in the art will recognize, with the appropriate dosage ultimately being at the discretion of the attendant physician. Dosage regimens may be adjusted to provide the desired response. Preferably, the growth factor of the present invention is administered at an amount and for a time such that permeability to BBB is increased, then at least one dosages of the therapeutic agent are administered subsequently to the subject to achieve an improved therapeutic response.

In some embodiments, the VEGF polypeptide can be administered about 15-180 minutes (e.g., 15-120, 15-90, 15-60, 30-120, 30-90, or 30-60 minutes) prior to the administration of the therapeutic agent or diagnostic agent. In some embodiments, the VEGF polypeptide is administered about 15, 20, 25, 30, 35, 40, 45 or 50 min prior to the administration of the therapeutic agent or diagnostic agent. In one example, the VEGF is administered about 45 minutes prior to the administration of the therapeutic agent. In another example, the administration of the VEGF is about 3 hours prior to the administration of the therapeutic agent or diagnostic agent.

In some embodiment, the treatment methods disclosed herein further comprise administering the subject one or more additional doses of the VEGF polypeptide after administration of the therapeutic agent (e.g., an anti-cancer agent) or the diagnostic agent. For example, a first additional dose of VEGF can be given to the subject about 2-24 hours (e.g., 2-12 hours, 3-8 hours, or 3-5 hours) after administration of the therapeutic/diagnostic agent. In one example, the first additional dose of VEGF is given to the subject about 3 hours after the administration of the therapeutic/diagnostic agent. In some examples, a second additional dose of VEGF can be given to the subject within a suitable window after administration of the first additional VEGF dose, for example, 2-24 hours after the first additional dose of VEGF (e.g., 2-12 hours, 3-8 hours, or 3-5 hours). In one example, the second additional dose of VEGF can be given to the subject about 3 hours after administration of the first additional dose of VEGF. When needed, further doses of VEGF can be given to the subject alone with treatment of the therapeutic agent or the diagnostic agent.

When multiple doses of VEGF are used, the dose of each VEGF administration may be the same. Alternatively, different VEGF doses may be given at different times. In some instances, a low dose of VEGF (e.g., within the range of the low doses disclosed herein) can be administered to a subject each time, while doses of VEGF administered at different times may be the same or may vary.

During the course of VEGF treatment, one or more additional doses of the therapeutic agent or the diagnostic agent may be administered to the subject following routine procedures of the agent. In some instances, each administration of the therapeutic or diagnostic agent may be given within a suitable window after the last administration of VEGF, for example, within 30 minutes to 3 hours, optionally about 45 minutes after the last administration of VEGF.

In some examples, a low dose of a VEGF polypeptide (e.g., VEGF165A) is administered to a subject such as a human patient. About 30-60 minutes (e.g., 45 minutes), an effective amount of a therapeutic agent or a diagnostic agent is administered to the same subject. The subject may be followed up with one or more low doses of VEGF afterwards, for example, a first additional low dose of VEGF 2-8 hours after the administration of the therapeutic/diagnostic agent, and optionally a second additional low dose of VEGF 2-8 hours after the first additional dose of VEGF. Additional doses of the therapeutic agent or the diagnostic agent may be given to the subject before and/or after the first additional dose of VEGF and optionally before and/or after the second additional dose of VEGF.

In some instances, more than 2 doses of VEGF is administered to a subject after the administration of a therapeutic agent or a diagnostic agent. For example, at least 2, 3, 4, 5, 6, 7, 8, 9, or 10 doses (e.g., low doses) of VEGF may be administered to a subject after administration of the therapeutic agent or the diagnostic agent. The doses of VEGF administered after the administration of the therapeutic agent may be administered consecutively (e.g., with no intervening administration of a therapeutic agent). Alternatively the doses of VEGF administered after the administration of the therapeutic agent may be administered non-consecutively (e.g., with intervening administration of a therapeutic agent).

In some instances, the time interval between doses of VEGF (e.g., consecutive or non-consecutive) is at most 4 hours (e.g., 15 minutes, 30 minutes, 1 hour, 1.5 hours, 2 hours, 3 hours, or 4 hours). The time interval between doses of VEGF (e.g., consecutive or non-consecutive) may be between 1 and 4 hours, between 2 and 4 hours, between 3 and hours, between 1.5 and 4 hours, between 2.5 and 4 hours, between 2 and 3 hours, or between 2.5 and 3.5 hours. In some instances, the time interval between doses of VEGF (e.g., consecutive or non-consecutive) is 3 hours.

In some examples, each dose of VEGF administered to a subject is the same amount. In some instances, at least two doses of VEGF administered to a subject are the same amount. In some instances, at least two doses of VEGF administered to a subject are different amounts. In some instances, all doses of VEGF administered to a subject are different amounts.

In some embodiments, the methods described herein can be applied for treating a brain disease such as a brain tumor in a subject. In some examples, the brain tumor is glioblastoma (e.g., glioblastoma multiform). The term “subject” or “patient” refers to an animal including the human species that is treatable with the method of the present invention. The term “subject” or “patient” intended to refer to both the male and female gender unless one gender is specifically indicated. Accordingly, the term “subject” or “patient” comprises any mammal which may benefit from the treatment method of the present disclosure.

The terms “tumor” and “cancer ” are used interchangeably herein, and is intended to mean any cellular malignancy whose unique trait is the loss of normal controls that results in unregulated growth, lack of differentiation and/or ability to invade local tissues and metastasize. Human brain tumors include, but are not limited to, gliomas, metastases, meningiomas, pituitary adenomas, and acoustic neuromas. Examples of gliomas include astrocytoma, pilocytic astrocytoma, low-grade astrocytoma, anaplastic astrocytoma, glioblastoma multiforme, brain stern glioma, ependymoma, subependymoma, ganglioneuroma, mixed glioma, oligodendroglioma, and optic nerve glioma. In some examples, the brain tumor is glioblastoma multiforme. Examples of non-glial tumors include acoustic neuroma, chordoma, CNS lymphoma, craniopharyngioma, hemangioblastoma, medulloblastoma, meningioma, pineal tumors, pituitary tumors, primitive neuroectodermal tumors (PNET), rhabdoid tumors, and schwannoma. Tumors that affect the cranial nerves include gliomas of the optic nerve, neurofibromas of 8th cranial nerve, neurofibromas of 5th cranial nerve. Benign tumors include arachnoid, dermoid, epidermoid, colloid, and neuroepithelial cysts and any other slow growing tumors. While primary brain tumors, like those described above, originate in the brain itself, metastatic brain tumors (secondary brain tumors that begin as cancer in another part of the body) are the most common brain tumors. Cerebral metastases can spread from primary cancers including, but not limited to, cancers originating in the lung, skin (melanoma), kidney, colon and breast.

The term “treatment” as used herein are intended to mean obtaining a desired pharmacological and/or physiologic effect, e.g., delaying or inhibiting cancer growth or ameliorating ischemic injury to an organ (e.g., brain). The effect may be prophylactic in terms of completely or partially preventing a disease or symptom thereof and/or therapeutic in terms of a partial or complete cure for a disease and/or adverse effect attributable to the disease. “Treatment” as used herein includes preventative (e.g., prophylactic), curative or palliative treatment of a disease in a mammal, particularly human; and includes: (1) preventative (e.g., prophylactic), curative or palliative treatment of a disease or condition (e.g., a cancer or heart failure) from occurring in an individual who may be pre-disposed to the disease but has not yet been diagnosed as having it; (2) inhibiting a disease (e.g., by arresting its development); or (3) relieving a disease (e.g., reducing symptoms associated with the disease).

An anti-cancer drug such as those described herein may be co-used with a VEGF (as well as another growth factor as described herein) following the disclosures provided herein. A low dose VEGF was found to increase the BBB permeability to not only small molecule drugs but also protein drugs/nanoparticles/stem cells. Accordingly, both small-molecule anti-cancer drugs and biologics can be co-used with VEGF as described herein to enhance the treatment efficacy of the brain tumor.

In some embodiments, the methods described herein can be applied for treating a brain disorder, including, but not limited to, brain stroke, a neuropsychiatric disorder, or a neurodegenerative disease. Examples of such brain diseases are provided herein. In some examples, stem cells such as MSCs can be co-used with VEGF (as well as other growth factors as described herein) for treating brain stroke or a neurodegenerative disease following the disclosures provided herein. In other instances, an anti-coagulant (e.g., those described herein) may be co-used with VEGF for treating brain stroke. Further, an anti-psychotic or anti-dementia agent, including any of those described herein, may be co-used with VEGF for treating a psychotic disorder or dementia. Examples of these target diseases are also provided in the present disclosure.

The term “stroke” as used herein is intended to mean any event that blocks or reduces blood supply to all or part of the brain. Stroke may be caused by thrombosis, embolism or hemorrhage, and may be referred to as ischemic stroke (including thrombotic stroke and embolic stroke and resulting from thrombosis, embolism, systemic hypo-perfusion, and the like) or hemorrhagic stroke (resulting from intracerebral hemorrhage, subarachnoid hemorrhage, subdural hemorrhage, epidural hemorrhage, and the like). As used herein, stroke excludes heat-stroke and transient ischemic attacks (TIA). Heat-stroke results from an elevated temperature in the body and its clinical manifestations in the brain are different from those of stroke as defined herein (i.e., interruption of blood supply associated with reduced oxygen in the brain). TIA are sometimes referred to as “mini-strokes,” however they can be distinguished from stroke as defined herein due to their ability to resolve completely within 24 hours of occurrence. Stroke is diagnosed through neurological examination, blood tests, and/or medical imaging techniques such as Computed Tomography (CT) scans (e.g., without contrast agents), Magnetic Resonance imaging (MRI) scans, Doppler ultrasound, and arteriography.

The term “neuropsychiatric disorder” is intended to mean a neurological disturbance that is typically labeled according to which of the four mental faculties are affected. For example, one group includes disorders of thinking and cognition, such as schizophrenia and delirium; a second group includes disorders of mood, such as affective disorders and anxiety; a third group includes disorders of social behavior, such as character defects and personality disorders; and a fourth group includes disorders of learning, memory, and intelligence, such as mental retardation and dementia. Accordingly, neuropsychiatric disorders of the present. disclosure encompass schizophrenia, delirium, Alzheimer's disease (AD), depression, mania, attention deficit disorders (ADD), attention deficit hyperactivity disorder (ADHD), drug addiction, mild cognitive impairment, dementia, agitation, apathy, anxiety, psychoses, post-traumatic stress disorders, irritability, and bipolar disorder.

The term “neurodegenerative disease” as used herein refers to a condition characterized by the death of neurons in different regions of the nervous system and the consequent functional impairment of the affected subjects. Neurodegenerative disease of the present disclosure encompasses Alzhemer's disease (AD), argyrophilic grain disease, amyotrophic lateral sclerosis (ALS), ALS-parkinsonism dementia complex of Guam, vascular dementia, frontotemporal dementia, semantic dementia, dementia with Lewy bodies, Huntington's disease, inclusion body myopathy, inclusion body myositis, or Parkinson's disease (PD).

In other embodiments, the methods described herein can be applied for brain imaging by co-use a VEGF (or other growth factors) with an imaging agent, such as a contrast agent. The contrast agent may be any agent that can be detected using computed tomography (CT) such as positron emission tomography (PET) or single photon emission computed tomography (SPECT); or magnetic resonance imaging (MRI). As a non-limiting example, the imaging agent may be a contrast agent for computed tomography (CT) or magnetic resonance imaging (MRI).

### Kits for Use in Treating or Diagnosing Brain Disorders

The present disclosure also provides kits for use in the methods described herein for treating or diagnosing a brain disease. Such kits can include at least two containers, one containing a first formulation that comprises a VEGF and a second formulation containing a second formulation that comprises a therapeutic agent (e.g., an anti-cancer agent) as those described herein or a diagnostic agent as also described herein (e.g., an imaging agent). In some instances, a kit comprises a third formulation containing a third formulation that comprises VEGF, wherein the third formulation may be for systematical administration to a subject in need of the treatment 2-4 hours after administration of the second formulation.

In some instances, the kit further comprises at least one (iv) fourth container containing a fourth formulation that comprises a vascular endothelial growth factor (VEGF) and wherein the fourth formulation may be for systematical administration to a subject in need of the treatment 2-4 hours after administration of the third formulation. In instances with more than one fourth container, the time interval between consecutive doses of VEGF may be 2-4 hours (e.g., the time interval may be 3 hours).

In some embodiments, the kit can comprise instructions for use in accordance with any of the methods described herein. The included instructions can comprise a description of administration of the VEGF and/or the therapeutic/diagnostic agent to treat or diagnose a target brain disease as described herein. The kit may further comprise a description of selecting an individual suitable for the treatment based on identifying whether that individual has the target disease. In still other embodiments, the instructions may comprise a description of administering the VEGF or the therapeutic/diagnostic agent to an individual at risk of the target disease.

The instructions relating to the use of a VEGF and/or the therapeutic/diagnostic agent generally include information as to dosage, dosing schedule, and route of administration for the intended treatment or diagnosis. The containers may be unit doses, bulk packages (e.g., multi-dose packages) or sub-unit doses. Instructions supplied in the kits described herein are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating/diagnosing, delaying the onset and/or alleviating a brain disease or disorder such as those described herein. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle).

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present invention will employ, unless otherwise indicated, conventional techniques of molecular biology (including recombinant techniques), microbiology, cell biology, biochemistry and immunology, which are within the skill of the art. Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Examples

### Inducing a Transient Increase in Blood Brain Barrier Permeability for Improved Liposomal Drug Therapy of Glioblastoma Multiforme

This study examines the effects of lose doses of human vascular endothelial growth factor A (VEGF-A) in enhancing permeability of the blood brain barrier (BBB), thereby facilitating delivery of therapeutic agents to the brain. It was reported herein that a lose dose of systemically injected human VEGF polypeptide induced a short period of increased BBB permeability in both mice and pigs. VEGF was found to increase brain delivery of a range of exemplary molecules, including nanoparticles with different sizes, small molecules, and liposomal chemotherapeutics. The results indicate that the window of BBB permeability induced by VEGF is transient and normal BBB integrity restored within four hours after administration of VEGF as observed in a mouse model. Combination of VEGF and liposomal doxorubicin unexpectedly extended animal survival in a mouse model of human glioblastoma. Surprisingly, no systemic toxicity was observed when VEGF was given to mice.

Accordingly, the instant results support that VEGF can be used to facilitate delivery of therapeutic agents such as nanoparticles or liposome agents across the BBB, thereby facilitating treatment of brain disorders.

### Materials and Methods

(i) Animal Experimentation

Animals used for drug biodistribution studies were 8-10 week-old male Friend leukemia virus B (FVB) mice, weighing approximately 25 g. 6-8 week old male BALB/c NU mice, approximately 21 g, were used for human GBM tumour xenograft experiments. For PDAC tumour xenografts, 8 week old NOD/SCID mice weighing 25-30 g were used, and 8-10 week old female ICR mice, approximately 30 g, were used for mechanistic and safety studies. All mice were purchased from BioLasco, Taiwan. Mice were housed in a 12 hour day-night cycle with free access to food and water. For large animal studies, Lanyu minipigs, 19-24 kg were used. All mouse experiments were approved by Academia Sinica Institutional Animal Care and Use Committee (IACUC) and pigs were used in accordance with approved protocols from Taiwan National Laboratory Animal Center, under supervision of veterinary staff.

(ii) Agent Administration

In mice, drugs were administered as a bolus injection by tail vein using a 0.30 G insulin needle, unless otherwise stated. Recombinant human VEGF165A (Peprotech, Taiwan) was suspended in 0.1% w/v bovine serum albumin and administered via lateral tail vein at a dose of 1.5 ng/g body weight, unless stated otherwise. Evans blue (Sigma E2129) was suspended at 4% w/v in normal saline and administered at a dose of 4 nal/kg. Doxorubicin Hydrochloride (Sigma D1515) suspended in saline, or LipoDox (TTY Bio, Taiwan) was administered slowly at doses between 2-8 mg/kg by lateral tail vein. Temozolomide (Sigma T2577) was administered at either 5 mg/kg or 20 mg/kg. Fluorescent PEG-modified yellow-green polystyrene microspheres with 20, 100 and 500 nm solid core diameters (Life Technologies, Thermo Fisher) were administered at a dose of 3 mg/kg. Lipopolysaccharide (Sigma L4391), used to induce neuroinflammation, was given at a dose of 5 mg/kg. In pigs, rhVEGF165A (0.2 μg/kg, 2 μg/ml) or vehicle control was injected into the right common carotid artery. LipoDox (TTY Bio, Taiwan), diluted to 0.35 mg/ml in 5% w/v dextrose, was administered by intravenous infusion by syringe pump at a dose of 1.5 mg/kg at a rate of approximately 3.0 ml/min Yellow-green PEG-modified polystyrene nanoparticles (100 nm core diameter) were administered by bolus injection at a dose of 3 mg/kg.

(iii) Nanoparticle PEGylation

Fluorescent nanoparticles were PEG-modified using mPEG amine (5 kD, Nanocs, Taiwan) and Carbodiimide (Sigma) and characterised using a Malvern ZetaSizer ZS, as previously described. Lundy et al., Sci. Rep., 6:25613 (2016).

(iv) Contrast Magnetic Resonance Imaging (MRI)

For mice, a Pharmascan 7T 16-cm bore horizontal system was operated by a technician. FVB mice, anaesthetised with inhaled isoflurane, were injected with VEGF or an equal volume of vehicle control. Pre-contrast T1-weighted spin echo images were taken (repetition time (TR)=400 ms, echo time (TE)=10.8 ms, field of view (FOV)=2×2 cm, number of excitations (NEX)=8, slice thickness 0.8 mm). 45 minutes, or 4 hours, after VEGF administration, contrast agent (Gadovist, Bayer) was administered at 0.2 mmol/kg by catheterised tail vein. Post-contrast T1-weighted images were acquired one minute after contrast agent injection. The SNR was calculated by dividing the signal of a ROI (mean pixel intensity) by the standard deviation of the background noise. All image acquisition, SNR measurement and tumour volume measurement was performed by two MRI operating technicians, who were blinded to the study groups.

For pigs, VEGF or vehicle control was administered and two pre-contrast T1-weighted turbo spin echo images were taken (TR=600 ms, TE=10 ms, FOV=20×20 cm, slice thickness=3 mm) using a Philips Achieva X 3.0 3T MRI machine. 45 minutes following VEGF administration, Gadodiamide (Omniscan, GE Healthcare) was administered intravenously by power injector at a dose of 0.1 mmol/kg (approximately 5 ml). One minute later, a series of three post-contrast images were taken at the same settings. The two pre-contrast images were averaged, and the post contrast image with the highest SNR from each animal was selected for analysis.

(v) Evans Blue Quantification

After 30 minutes circulation, 50 ml phosphate buffered saline (PBS) was perfused through the abdominal aorta. Organs were removed, cut into multiple smaller pieces, rinsed, dried, weighed, homogenised in 500 μl formamide, heated at 60° C. overnight and centrifuged at 21,000×g for 15 minutes. Supernatant absorbance was measured at 620 nm with background correction at 740 nm, and unknowns quantified by standard curve. Organs from mice which did not receive Evans blue injection were used to establish blank absorbance readings. Radu et al., J. Vis. Exp., No. 73, e50062 (2013).

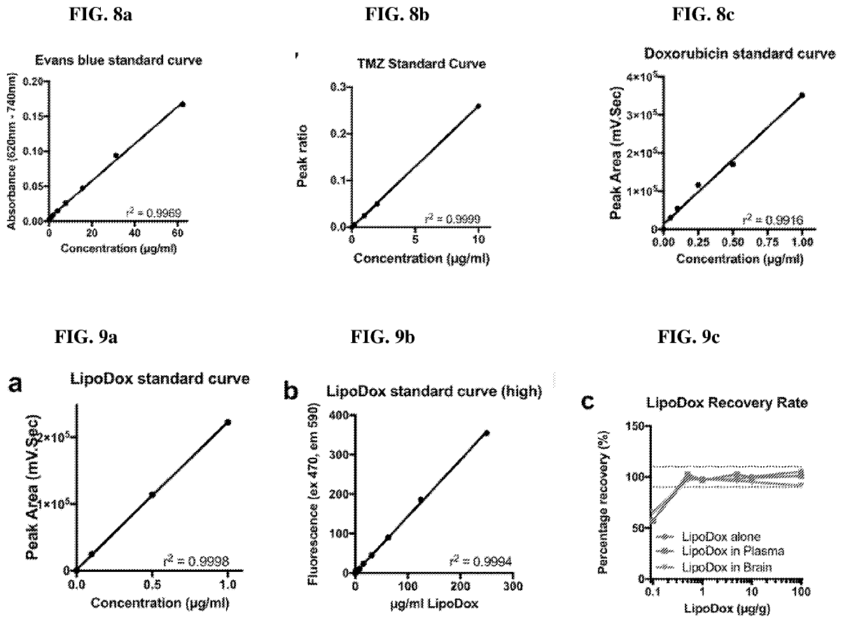

Concentrations from multiple pieces of the same organ were averaged. The standard curve for this method is shown in FIG. 8a. For imaging of Evans blue extravasation, a method from del Valle and colleagues was adapted. del Valle et al., J. Neurosci. Methods, 174 (1), 42-49 (2008). Mice were intracardially perfused with 50 ml PBS followed by 50 ml of Evans blue (1% w/v) in paraformaldehyde (4% w/v). Cryolesion injury, to induce a local area of BBB damage, was used as a positive control. The brain was embedded in optimal cutting temperature compound (OCT) and cryosectioned into 20 μm thick sections for analysis.

(vi) Fluorescent PEG-Modified Polystyrene Nanoparticle Quantification

In mice, nanoparticles were allowed to circulate for 30 minutes before animals were perfused, as described above. IVIS was used to quantify nanoparticle retention (ex 485, em 530 nm). A brain from a mouse which did not receive nanoparticle injection was used to correct for background. In pigs, HPLC was used to quantify nanoparticle retention. Chen et al., Nanoscale, 7 (38), 15863-15872 (2015). Briefly, nanoparticle fluorescent dye was extracted into o-xylene, and quantified using a Waters e2695 separation module and X-bridge C18 (250×4.6 mm, 5 μm) column with a mobile phase of 77:23 methanol:water, flow rate 1 ml/min Detection used a Waters 2475 FLR detector with excitation at 505 nm and emission at 515 nm.

(vii) HPLC Quantification of Temozolomide (TMZ)

Approximately 100 mg tissue was homogenised in acidified ammonium acetate (200 μl, 10 mM pH 3.5), zinc sulphate (200 μl, 100 mM) and methanol (400 μl), followed by centrifugation at 10,000×g for 30 minutes at 4° C. Supernatant was taken for HPLC analysis. Separation was carried out with a Water e2695 separation module using 80:20 ratio of acetic acid (0.1% v/v) to methanol at a flow rate of 0.8 ml/min in an Atlantis T3 3 μm HPLC column at 35° C. Detection was performed using a Waters 2489 UV/vis detector at 316 nm. Theophylline was used as an internal standard, measured at 275 nm, and results calculated as the peak ratio of TMZ to theophylline. Unknowns were calculated from a standard curve of TMZ dissolved in lysis buffer, as shown in FIG. 8b.

(viii) HPLC Quantification of Doxorubicin and Liposomal Doxorubicin

Organs were removed, dried, weighed, cut into multiple smaller pieces (approximately 100 mg tissue or 100 μl plasma) then thoroughly homogenised with 1 ml lysis buffer (0.25 M sucrose, 5 mM Tris-HCl, 1 mM MgSO4, 1 mM CaCl2, pH 6.7). 200 μl homogenate was then mixed with 1 ml acidified alcohol (70% ethanol, 0.3 N HCl), left overnight at −20° C., then centrifuged at 10,000×g for 30 minutes at 4° C. Supernatant was taken for HPLC analysis. Separation was via a Waters e2695 separation module, mobile phase 35% 10 mM KH2PO4, 65% methanol, flow rate 1 ml/min in an X-Bridge 5 μm column at 40° C. Detection used a Waters e2475 module (ex 480, em 600 nm).

For LipoDox extraction, the protocol was modified to include 30 minutes of sonication, 2 rounds of freeze thaw, and the addition of Triton-X100 (1% v/v) to the lysis buffer, adapted previous publications. Kovacs et al., J. Control. Release, 187, 74-82 (2014); and Laginha et al., Clin. Cancer Res., 11 (19 Pt 1), 6944-6949 (2005). Standard curves, example HPLC peaks and recovery rates are shown in FIGS. 9a-9d.

(ix) Glioblastoma Multiform (GBM) Xenograft Models

DBTRG-05MG human glioblastoma cells were used in this study. Evidence of luciferase expression is shown in FIG. 13a. DBTRG-05MG cells were routinely cultured at 37° C. in RPMI 1640 media supplemented with 10% FBS, 1 mM sodium pyruvate and 1% penicillin/streptomycin. MTT assay was carried out in accordance with the manufacturer protocol. For GBM tumour generation, 300,000 live DBTRG-05MG cells, suspended in 6 μl sterile saline, were administered to 6 week old BALB/c NU mice by stereotactic injection, 0.5 ml/min The location was 2 mm posterior to the bregma, 1.5 mm laterally in the right cerebral hemisphere, and 2.5 mm deep from the dura, thus delivering the cells into the thalamus. Baumann et al., J. Vis. Exp., No. 67, 3-6 (2012). Bone wax was applied to the skull and the skin was closed with sutures. The success rate of xenograft tumour formation was approximately 90%. Animal deaths were recorded when the animal succumbed from tumour progression, or upon sacrifice if they met pre-determined criteria including severe cachexia (loss of >25% starting body weight), inability to feed, and lack of response to toe-pinch stimulus. Gholamin et al., Cureus, 4, 1-14 (2013). Further characterisation of the tumour model is shown in FIGS. 13A-13C.

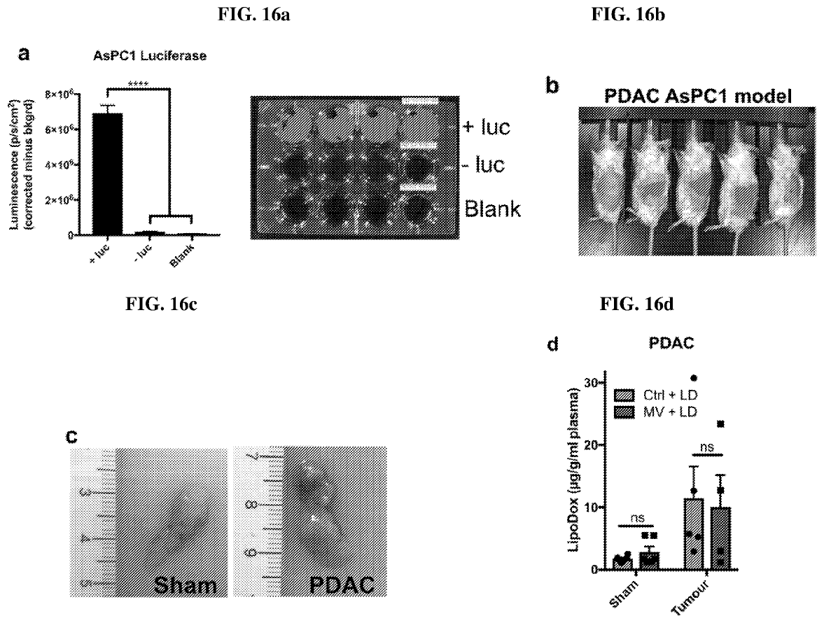

For subcutaneous GBM xenografts, 1×10⁶ DBTRG-05MG cells were injected into each flank of balb/c NU mice and allowed to grow for 58 days. For orthotopic model of pancreatic cancer (PDAC), luciferase-expressing AsPC1 human pancreatic cancer cells, gifted by Dr. Yu-Wen Tien, National Taiwan University Hospital, Taiwan, were routinely cultured at 37° C. in RPMI 1640 with 10% FBS, and 1% penicillin/streptomycin. Tan et al., Tumour Biol., 6 (1), 89-98 (1985). 5×10⁵ live AsPC1 cells, suspended in 10 μl sterile PBS, were administered into the pancreas. Chai et al., J. Vis. Exp. 2013, No. 76, 2-6 (2013). After returning the pancreas to the abdominal cavity, the incision was closed in two layers using a continuous suture for the peritoneum and an interrupted suture for the skin. Further information is available in FIGS. 16A-16D.

(x) IVIS Assessment of Tumour Progression

Luciferin substrate, 75 μg/g, (Monolight, BD Bioscience) was given by intraperitoneal injection. Mice were anaesthetised with inhaled isoflurane and repeated IVIS images were acquired at five minute intervals using a Perkin Elmer IVIS Spectrum. The time point presenting the strongest luminescent signal was selected for analysis. Background readings from a sham mouse present in every frame were subtracted.

(xi) Immunofluorescence Staining

Brain tumours from mice which died between day 60 to day 70 were selected for immunofluorescence staining. VEGF+Ctrl samples were included for reference, although those mice died earlier than day 60. Primary antibodies and dilutions used were anti-Ki67 (1:500 GeneTex GTX16667), Isolectin IB4-AlexaFluor 647, anti-GFAP (1:500 AbCam ab68428), anti-Iba1 (1:1000 Wako 019-19741), anti-p-glycoprotein (1:100 AbCam ab170904), anti-pdgfrβ (1:100, ab32570 Abcam), anti-claudin-5 (1:50 34-1600 Thermo Fisher Scientific), anti-CD31 (1:100 550274, BD Pharmingen). Samples were fixed in 4% w/v paraformaldehyde (PFA) pH 6.8 overnight, embedded in paraffin and sectioned. Slides were de-waxed with xylene, rehydrated through graded alcohols to water, then subjected to antigen retrieval, permeabilisation and blocking in accordance with the manufacturers' instructions. Primary antibody was applied overnight at 4° C. diluted in blocking buffer, apart from isolectin, which was applied for one hour at room temperature. Slides were washed 3× with PBS, before the second primary antibody was added and left overnight at 4° C. Secondary antibodies used were goat anti-rabbit IgG-AlexaFluor 568 (Invitrogen A-11011) and goat anti-rat IgG-AlexaFluor 488 (Invitrogen A-11006), applied for one hour at room temperature. At least three separate sections were examined, and at least three images were captured per section. For frozen sections, samples were perfusion fixed then submerged in 4% paraformaldehyde (PFA), cryoprotected in 30% w/v sucrose solution, then frozen in OCT, sectioned and stained as above, continuing on from blocking. H&E staining (Mayers) followed standard lab protocols. Images were captured using a Zeiss AxioScop microscope with AxioFluor objective lenses and AxioVision software, or a Model-Zeiss LSM 700 Stage confocal microscope.

(xii) Transmission Electron Microscopy

Mice were anaesthetised and perfused with PBS followed by 100 ml 4% PFA in 0.1 mM phosphate buffer, pH 7.4. The brain was removed and post-fixed in 4% PFA (overnight, 4° C.) and washed in PBS. Coronal brain sections (100 μm thick) were cut on the same day with a cryomicrotome and processed free floating. Sections were immersed in 4% PFA, 2.5% glutaraldehye in PBS (overnight, 4 ° C.), washed with PBS for 5 minutes for 3 times. The specimens were immersed in 1% osmium tetroxide for 45 minutes, dehydrated and embedded with Spurr's low viscosity resin. The sample was then trimmed and sectioned using a Leica EM UC6 ultramicrotome. The ultrathin sections were then double stained with uranyl acetate and lead citrate. Images were acquired using Jeol JEM 1200EX TEM with an acceleration voltage of 80 KV.

(xiii) Enzyme Linked Immunosorbent Assay (ELISA)

For plasma S100β, mouse plasma was separated by 15 minutes centrifugation at 1,500×g and the ELISA was carried out according to the manufacturer's instructions (Elabscience, E-EL-M1033). Diluted brain homogenate in saline was used as a positive control. To measure plasma rhVEGF, an anti-human VEGF ELISA kit (Boster, EK0539) was used, following the manufacturer protocol. Samples from the same mice prior to VEGF administration were used as blanks.

(xiv) Real Time Quantitative PCR

Samples of mouse cerebral cortex weighing approximately 50 mg were homogenised in Trizol, and total RNA extracted via the manufacturer's protocol, then quantified by Nanodrop. Samples were reverse transcribed to cDNA using a SuperScript III Reverse-Transcriptase Kit (Life Technology). OmicsGreen qPCR SYBR Green master mix (Omics Bio, Taiwan) was used to monitor amplification using an Applied Biosystems 7500 Real-Time PCR system. GAPDH was used an internal control. Primers used are shown in Table 1.

TABLE 1
PCR Primers

| | | | | | | C_{T} |
|---|---|---|---|---|---|---|
| Gene name | Name. | | Forward | Reverse | Product | (brain |
| (human/mouse) | Function | Gene ID | sequence | sequence | length | control) |
| GAPDH/Gapdh | GAPDH. | NM_001289726.1 | ACCCAGAAGAC | CACATTGGG | 171 | 18 |
| | Internal | | TGTGGATGG | GGTAGGAAC | | |
| | control | | (SEQ ID | AC (SEQ | | |
| | | | NO: 2) | ID NO: 3) | | |

Neuroinflammation markers

| | | | | | | |
|---|---|---|---|---|---|---|
| TNF/Tnf | TNFα. Acute | NM_013693 | GACCCTCACAC | CCTCCACTT | 80 | 28 |
| | phase | | TCAGATCTTCT | GGTTTGCT | | |
| | cytokine | | (SEQ ID | (SEQ ID | | |
| | | | NO: 4) | NO: 5) | | |
| IL-1b/Il1b | IL-1β. | NM_008361 | TGCCACCTTTT | ATGTGCGAG | 136 | 31 |
| | Inflammatory | | GACAGTGATG | ATTTG | | |
| | cytokine | | (SEQ ID | (SEQ ID | | |
| | | | NO: 6) | NO: 7) | | |
| IL6/Il6 | IL-6. Acute | NM_031168 | TCCAGAAACCG | CACCAGCAT | 73 | 31 |
| | phase | | CTATGAAGTTC | CAGTCCCAA | | |
| | cytokine | | (SEQ ID | GA (SEQ | | |
| | | | NO: 8) | ID NO: 9) | | |
| CCL2/ccl2 | CCL2. | NM_011333 | GTTGGCTCAGC | AGCCTACTC | 81 | 30 |
| | Chemokine | | CAGATGCA | ATTGGGATC | | |
| | | | (SEQ ID | TTG (SEQ | | |
| | | | NO: 10) | ID NO: 11) | | |
| GFAP/Gfap | GFAP. | NM_001131020.1 | GAACAACCTGG | GCGATTCAA | 80 | 29 |
| | Astrocyte | | CTGCGTATAG | CCTTTCTCT | | |
| | marker | | (SEQ ID | CCAA (SEQ | | |
| | | | NO: 12) | ID NO: 13) | | |
| CXCL1/cxcl1 | CXCL1. | NM_008176 | CACCCAAACCG | AATTTTCTG | 82 | 21 |
| | Chemokine | | AAGTCATAGC | AACCAAGGG | | |
| | | | (SEQ ID | AGCTT | | |
| | | | NO: 14) | (SEQ ID | | |
| | | | | NO: 15) | | |
| FN1/Fn1 | Fibronectin 1 | NM_010233 | AGGCAATGGAC | CTCGGTTGT | 104 | 24 |
| | | | GCATCAC | CCTTCTTG | | |
| | | | (SEQ ID | (SEQ ID | | |
| | | | NO: 16) | NO: 17) | | |
| IL-1a/Il1a | IL-1α. Acute | NM_010554 | CGCTTGAGTCG | CAGAGAGAG | 115 | 31 |
| | phase | | GCAAAGAAATC | ATGGTCAAT | | |
| | cytokine. | | (SEQ ID | GGCA (SEQ | | |
| | | | NO: 18) | ID NO: 19) | | |

BBB components

| | | | | | | |
|---|---|---|---|---|---|---|
| TFR/Tfrc | Transferrin | NM_011638 | CTGCTCATCAC | TGACCCCAT | 108 | 27 |
| | receptor | | TATGGTGGCTA | GGCAAAACT | | |
| | | | (SEQ ID | GA (SEQ | | |
| | | | NO: 20) | ID NO: 21) | | |
| CRT/Slc6a8 | Creatinine | NM_133987.2 | GTGGGGGTAAG | GCCACAACT | 103 | 33 |
| | transporter | | GGTGGAATGTA | ACACACTCC | | |
| | | | (SEQ ID | CAA (SEQ | | |
| | | | NO: 22) | ID NO: 23) | | |
| GLUT1/Slc2a1 | Glucose | NM_011400.3 | TGGCGGGAGAC | GCCCGTCAC | 110 | 24 |
| | transporter | | GCATAGTTA | CTTGCT | | |
| | | | (SEQ ID | (SEQ ID | | |
| | | | NO: 24) | NO: 25) | | |
| ATA2/Slc38a2 | Amino acid | NM_175121.4 | ACGAAACAGAC | AAGCCCAAG | 92 | 23 |
| | transporter | | TTTCATCCAGG | GATTCCACT | | |
| | | | TA (SEQ ID | GC (SEQ | | |
| | | | NO: 26) | ID NO: 27) | | |
| MRP4/Abcc4 | Multidrug | NM_001163676.1 | GGGCGAGATGC | GGGTTGAGC | 93 | 30 |
| | resistance | | TGCCG (SEQ | CACCAGAAG | | |
| | pump | | ID NO: 28) | AA (SEQ | | |
| | | | | ID NO: 29) | | |
| MDR1a/Abcb1a | P- | NM_011076.2 | CCATCTTCCAA | CCATCACGA | 107 | 26 |
| | glycoprotein | | GGCTCTGCT | CCTCACGTG | | |
| | efflux pump | | (SEQ ID | TC (SEQ | | |
| | | | NO: 30) | ID NO: 31) | | |
| ZO1/Trp1 | Tight | NM_009386.2 | CCTGTGAAGCG | CGCGGAGAG | 100 | 25 |
| | junction | | TCACTGTGT | AGACAAGAT | | |
| | protein | | (SEQ ID | GT (SEQ | | |
| | | | NO: 32) | ID NO: 33) | | |
| ZO2/Tjp2 | Tight | NM_001198985 | GAGATGCCGGT | TTTGGAATC | 126 | 27 |
| | junction | | GCGGG (SEQ | CTTCTGCAG | | |
| | protein | | ID NO: 34) | GG (SEQ | | |
| | | | | ID NO: 35) | | |
| OCLN/Ocln | Tight | NM_008756.2 | CATAGTCAGAT | ATTTATGAT | 91 | 26 |
| | junction | | GGGGGTGGA | GAACAGCCC | | |
| | protein | | (SEQ ID | CC (SEQ | | |
| | | | NO: 36) | ID NO: 37) | | |
| CLDN5/Cldn5 | Tight | NM_013805.4 | GTCACGATGTT | AAATTCTGG | 106 | 25 |
| | junction | | GTGGTCCAG | GTCTGGTGC | | |
| | protein | | (SEQ ID | TG (SEQ | | |
| | | | NO: 38) | ID NO: 39) | | |
| JAM-A/Fur | Tight | NM_172647.2 | AGTGTACACCG | TGTAACTGT | 106 | 27 |
| (CD321) | junction | | AACCCTTGC | AATGGGCAC | | |
| | protein | | (SEQ ID | CG (SEQ | | |
| | | | NO: 40) | ID NO: 41) | | |
| SPARC1/ | ECM | NM_010097.4 | ACCTCTCCGCA | GGTGTCACC | 136 | 20 |
| Sparcl1 | adhesion | | GATCTAGCCA | AGTGTTGCA | | |
| | | | (SEQ ID | GT (SEQ | | |
| | | | NO: 42) | ID NO: 43) | | |
| MAOB/Maob | Enzymatic | NM_172778.2 | GCTGGACCAAA | TGGTTGTAC | 123 | 26 |
| | barrier | | TCTACAAAGCA | CTCCACACT | | |
| | | | (SEQ ID | GC (SEQ | | |
| | | | NO: 44) | ID NO: 45) | | |

(xv) Software and Statistics

GraphPad Prism 7.0b (Mac) was used for all statistical analysis and graph generation. For before-after analyses, paired t-test was used, and for grouped analyses one or two-way ANOVA (analysis of variance) with Tukey's post-test to correct for multiple comparisons were used. For tumour survival analyses, deaths were recorded and used to generate Kaplan-Meier survival curves which were compared using Mantel-Cox log rank tests. IVIS images of tumour luminescence and nanoparticle fluorescence were quantified using Living Image 4.0 software for Mac. MRI DICOM images were sorted in MicroDicom (Windows) and SNR calculation was performed in FIJI/ImageJ (Mac) using the measure tool. For heatmap generation, voxels within the animal were compared to the average of a 64*64 voxel region in the corner of the frame and the difference was scaled from 0 to 100, using Python. Adjustments to immunofluorescence image brightness and contrast were made to improve visual clarity and were applied equally to all images within a series. For colocalisation analysis, the raw images were analysed in Zeiss ZEN software. The threshold for no colocalisation was established using the DAPI/CD31 channels, and that threshold was then applied to the other channels. Pericyte alignment analysis was carried out using the freehand line selection tool in ImageJ. Figures were assembled in Affinity Designer (Mac).

(xvi) Antibody Administration Method

30 μl Anti-NrCAM primary antibody (abCam) was injected via tail vein, 45 minutes after VEGF or control administration. The antibody was allowed to circulate for 2 hours, then mice were perfused with 50 mL saline followed by 50 mL paraformaldehyde (4% w/v). The brain was removed, kept in 4% PFA overnight, then processed for frozen sections. As a positive control, 5 μl of antibody was injected directly into the brain prior to perfusion. Frozen sections were then stained using secondary antibody conjugated to Alexa 488. As a negative control, brain sections from an untreated animal were used. As a second positive control, a brain section from an untreated animal was stained with anti-nrCAM using conventional lab techniques (1hr room temperature). All images, aside from the stained positive control, were taken at fixed exposure lengths. The intensity of the green channel was quantified in ImageJ.

### Results

### (i) Low-Dose VEGF Induces a Transient Increase in BBB Permeability

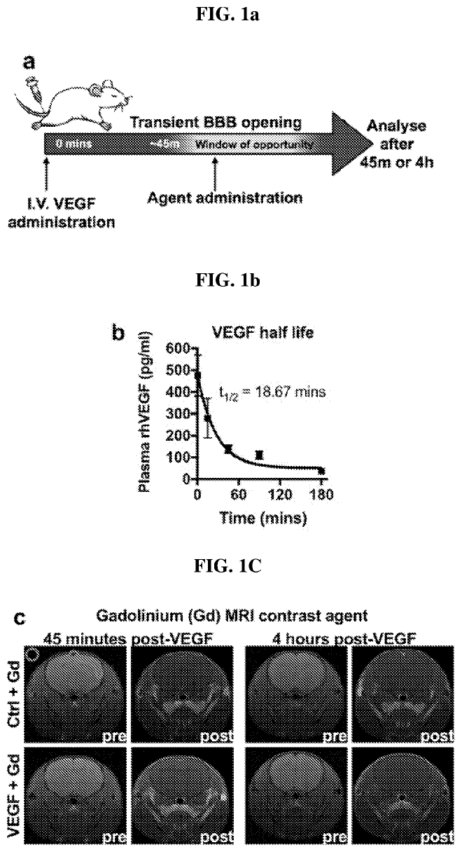

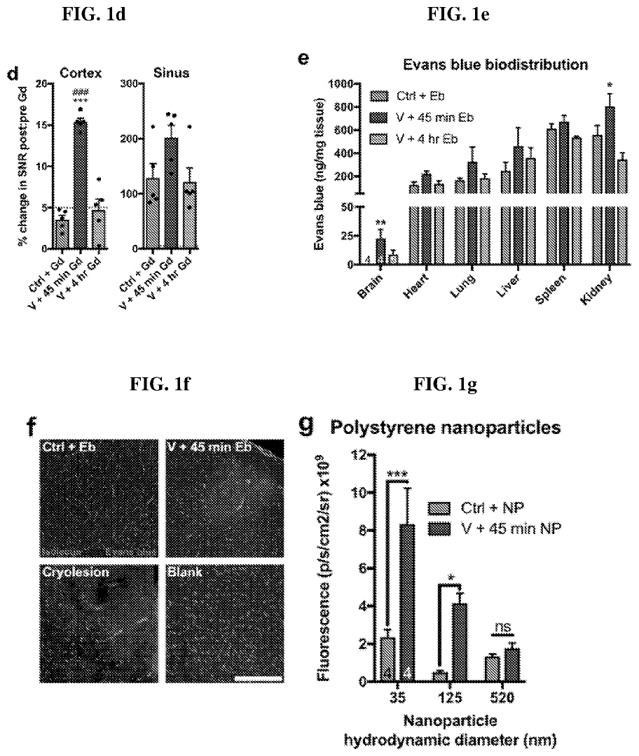

An exemplary experimental design is shown in FIG. 1a. Mice were intravenously injected with VEGF or vehicle control, followed by an agent either 45 minutes or 4 hours later. FIG. 1b shows the half-life of human VEGF in the mouse blood stream to be approximately 18.67 minutes.

Penetration of contrast agent into brain parenchyma, measured by magnetic resonance imaging (MRI), is often used to demonstrate BBB integrity in live animals. Burgess et al., Expert Opin. Drug Deliv., 11 (5), 711-721 (2014); Jiang et al., PLoS One, 9 (2) (2014); and Zheng et al., Biomaterials, 66, 9-20.e86407 (2015). Under normal conditions, only a small amount of Gadolinium-based agent (Gd) would be expected to enter the brain parenchyma, providing little contrast enhancement. As shown in FIGS. 1c and 1d, control-administered animals showed an average increase of only 3.5% in the signal to noise ratio (SNR) of cortex tissue in T1-weighted post-contrast images compared to pre-contrast images. However, when Gd was given 45 minutes following VEGF administration, there was a significant increase (16%, p<0.001) in the average SNR of the cortex, indicating that VEGF pre-treatment increased the penetration of Gd into the brain tissue. On the other hand, when Gd was given 4 hours after VEGF, the SNR enhancement remained less than 5%, indicating that BBB integrity had normalised (p=0.6150 vs. control, p<0.001 vs. VEGF 45 mins) Analysis of the area surrounding the central cerebral sinus (yellow boundary) showed a large signal enhancement in all groups due to contrast agent present in the sinus, with no significant difference between the groups. Example images are shown, indicating the regions of interest (ROIs) of random noise (red circle, left corner), central cerebral sinus (yellow circle in the middle), and cortex (blue curves at the right side of the yellow circle).

It was known in the art that the Evans blue dye can rapidly bind to serum albumin and does not cross the intact BBB. Huang et al., Adv Mater, 1-7 (2014); Bing et al., J. Ther. Ultrasound, 2 (1), 13 (2014); and Cardoso et al., Brain Res. Rev., 64 (2), 328-363 (2010). Evans blue was injected either 45 minutes or 4 hours after VEGF, and allowed to circulate for 30 minutes. As shown in FIG. le, VEGF pre-treated mice had a 4.85-fold higher concentration of Evans blue in the brain tissue compared to control-treated animals (p=0.0069), whereas when Evans blue was injected 4 hours following VEGF, no increase was detected (p=0.9405 vs. control). This again indicates that the increase in BBB permeability appears to be temporary.

The kidney also showed an increase in Evans blue uptake at 45 minutes. The standard curve for Evans blue quantification is shown in FIG. 8a. Representative sections of the brain cortex, shown alongside FIG. lf, show increased Evans blue staining (red) outside of isolectin-stained blood vessels (green). A positive control was carried out using cryolesion to cause local damage to the BBB prior to Evans blue injection. The lesioned area showed strong Evans blue signal in the parenchyma.

It is well established that smaller nanoparticles pass more readily into the brain than larger nanoparticles. Lin et al., Sci. Transl. Med., 4 (146), 146ra109-146ra109 (2012); Koffie et al., Proc. Natl. Acad. Sci. 2011, 108 (46), 18837-18842 (2011); and Ben-Zvi et al., Nature, 509 (7501), 507-511 (2014). To investigate the effect of VEGF on large particles for bypassing the BBB, PEG-modified polystyrene nanoparticles containing fluorescent dye were administered by tail vein injection 45 minutes following VEGF administration. The nanoparticles had solid core diameters of 20 nm, 100 nm and 500 nm, with hydrodynamic diameters of 52, 120 and 512 nm respectively, and neutral zeta potentials. Exemplary properties of the nanoparticle are shown in Table 2.

TABLE 2
Properties of polystyrene nanoparticles with carboxyl (COOH) surface
chemistry and following polyethylene glycol modification (PEG).

| Size | Surface | Diameter | Diameter | Zeta potential | |
|---|---|---|---|---|---|
| (nm) | chemistry | TEM (nm) | DLS (nm) | (mV) | PDI |
| 20 | COOH | 25.7 ± 2.4 | 34.6 ± 0.8 | −34.1 ± 2.0 | 0.08 |
| 20 | PEG | 28.2 ± 1.8 | 52.4 ± 8.9 | −1.0 ± 4.0 | 0.06 |
| 100 | COOH | 92.7 ± 2.1 | 105.4 ± 2.7 | −43.1 ± 2.2 | 0.04 |
| 100 | PEG | 95.0 ± 2.1 | 120.1 ± 4.0 | −1.4 ± 0.3 | 0.06 |
| 500 | COOH | 471.8 ± 5.3 | 471.8 ± 5.3 | −48.7 ± 0.3 | 0.05 |
| 500 | PEG | 482.8 ± 4.5 | 512.1 ± 6.0 | −1.2 ± 0.2 | 0.03 |

In Table 2, solid core diameter was measured by transmission electron microscopy (TEM) and hydrodynamic diameter and zeta potential were measured using a Malvern Zetasizer. Numbers show the mean±standard deviation. After allowing 30 minutes for nanoparticle circulation, mice were perfused with 50 ml saline and brain nanoparticle content was quantified by IVIS. As expected, 20 nm nanoparticles displayed more penetration into the brain than 100 nm or 500 nm nanoparticles under normal conditions. In VEGF-primed animals, a significant increase in 20 nm nanoparticle retention (3.5-fold vs. control, p=0.0002) by the brain was detected, as shown in FIG. 1g. A significant increase in 100 nm nanoparticle penetration (8-fold vs. control, p=0.0182) was also detected, but there was no significant change in the retention of 500 nm nanoparticles (p=0.9762 vs. control). In addition, preliminary evidence was also collected that show that VEGF pre-treatment allows the passage of systemically injected IgG antibody into the brain (FIG. 7), which could be beneficial for antibody-based chemotherapy.

In summary, these data show that a low-dose intravenous VEGF injection can increase BBB permeability and allow penetration of small molecules or nanoparticles into the brain. This effect appears transient, with restoration of BBB function 4 hours after the VEGF injection.

(ii) VEGF Enhanced the Permeability of Blood-Brain Barrier to Anti-Cancer Drugs

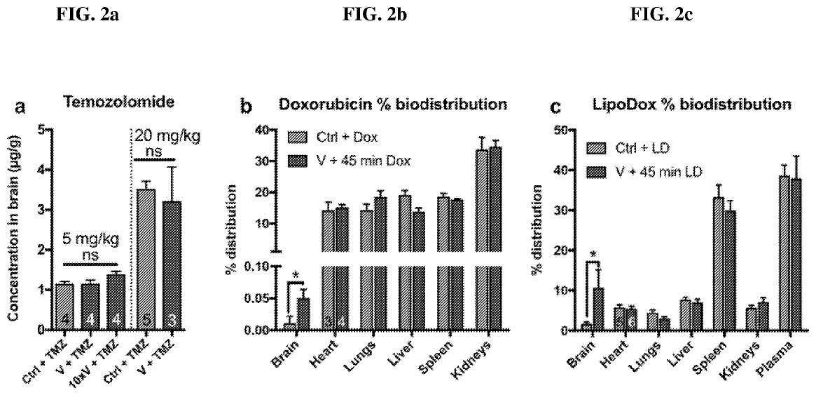

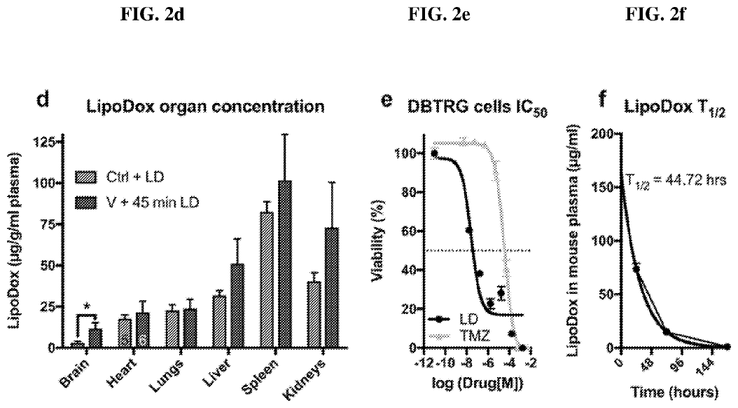

Next, it was sought to determine whether this same approach could be used to deliver different types of therapeutic compounds to brain tissue. Temozolomide (TMZ) is the first line drug therapy for treatment of GBM. As shown in FIG. 2a, VEGF does not significantly increase TMZ concentration in the brain, even using a 10-fold higher concentration of VEGF. Increasing the systemic dose of TMZ from 5 mg/kg to 20 mg/kg increased the amount of TMZ in the brain, but pre-treatment with VEGF did not further enhance brain concentration of TMZ. This may due to the fact that as a small molecule (MW=194.15) and highly lipid-soluble molecule, TMZ can penetrate into the brain for clinical effects on its own. Ostermann et al., Clin Cancer Res, 10 (11), 3728-3736 (2004). The standard curve and sample high performance liquid chromatography (HPLC) peaks for TMZ quantification are shown in FIG. 8b.

The effect of VEGF on BBB penetration of larger, water soluble molecules was then investigated, using Doxorubicin hydrochloride (MW=579.98) as an example. Doxorubicin has extremely poor entry into the brain following systemic administration. Many attempts have been made to deliver Doxorubicin to brain tumours due to its potent efficacy against other solid tumours. Aryal et al., J. Control. Release, 204, 60-69 (2015); Kovacs et al., 2014; and Wohlfart et al., J Control Release, 154 (1), 103-107 (2011). In this study, mice were injected with VEGF or a control followed by Doxorubicin (8 mg/kg) 45 minutes later. The drug was allowed to circulate for two hours before the animal was perfused with saline. Doxorubicin was then extracted from the vital organs and quantified by HPLC (FIG. 8c). Biodistribution results, shown in FIG. 2b, confirm that less than 0.1% of systemic Doxorubicin entered the brain of healthy control mice. Pre-treatment of VEGF resulted in a statistically significant increase (p=0.0180 vs. control) in the doxorubicin concentration in brain, although the distribution of doxorubicin in brain is still much lower than the distribution of this compound in other organs. FIG. 2b.

VEGF was then investigated for its effect in facilitate brain delivery of PEG-modified liposomal doxorubicin (LipoDox). It was determined that these liposomes are neutrally charged (−1.53 mV), with an average hydrodynamic diameter of 95.55 nm. See Table 3 below (numeric values represent mean±standard deviation as measured by a Malvern Zetasizer). LipoDox showed similar properties as the PEG-modified nanoparticles disclosed herein, which successfully entered the brain (FIG. 1e).

TABLE 3
Properties of LipoDox

| Size (nm) | Zeta potential (mV) | PDI |
|---|---|---|
| 95.55 ± 30.16 | −1.53 | 0.180 |

Results in FIG. 2c show a significant increase (6.4-fold vs. control, p=0.0037) in LipoDox entry into the brain following VEGF administration, showing that LipoDox was able to cross the healthy BBB in the presence of VEGF. FIG. 2d shows the data normalised against the blood plasma LipoDox concentration of each individual mouse at the time of sample collection, thus correcting for individual differences in drug metabolism and excretion. There were no significant differences detected in the concentration of LipoDox in any peripheral organs. FIGS. 9a-9d show the results of LipoDox quantification as determined by the HPLC method.

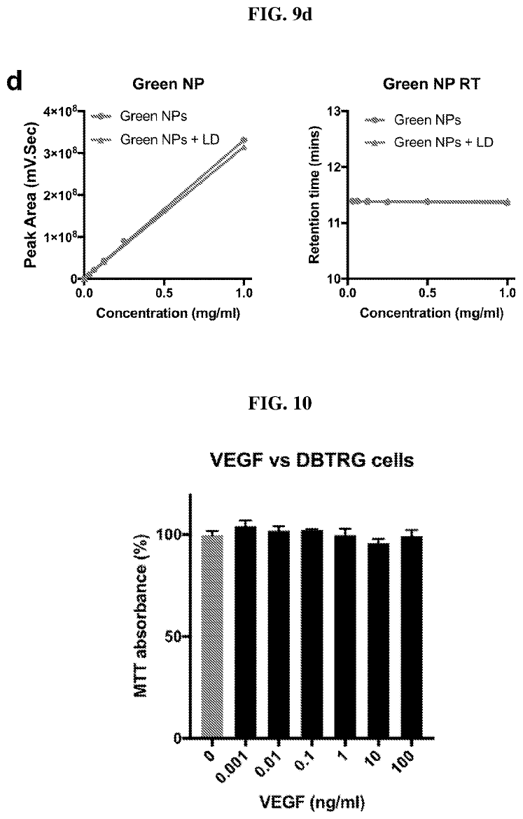

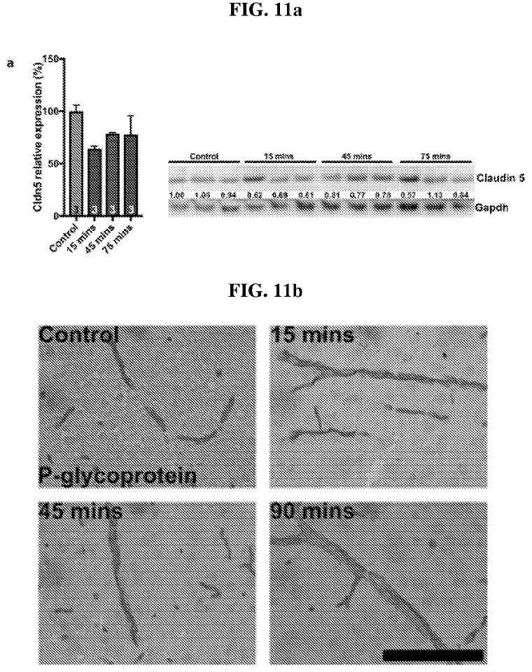

Using an MTT assay, it was found that LipoDox had a 25-fold lower IC₅₀ than TMZ when cultured with the human glioblastoma cell line DBTRG-05MG. FIG. 2e. In addition, it was determined the circulatory half-life of LipoDox to be 44.72 hours in mice, following systemic administration of a 5 mg/kg dose. FIG. 2f. This is significantly longer than the half-life of TMZ (1.8 hours) or doxorubicin (11 hours). Agarwala et al., Oncologist, 5 (2), 144-151 (2000); and Johansen et al., Cancer Chemother. Pharmacol., 5 (4), 267-270 (1981). It was also found that VEGF did not affect DBTRG-05MG cell viability at any given concentration (FIG. 10).

(iii) VEGF Enhances Drug Delivery to the Brain in a Large Animal Model

The effect of VEGF in facilitating drug delivery to the brain has further been investigated in a pig model to confirm that the results reported herein could be scaled up to more clinically relevant drug doses.

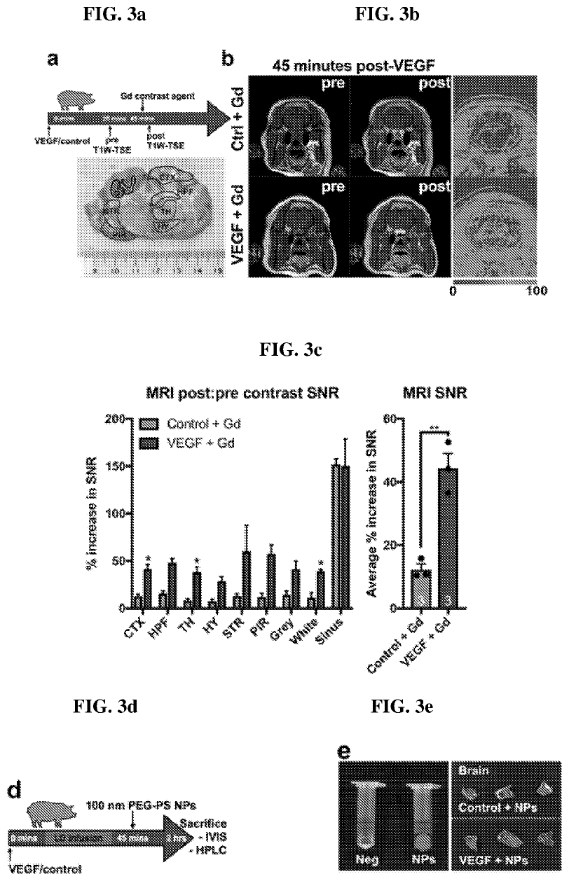

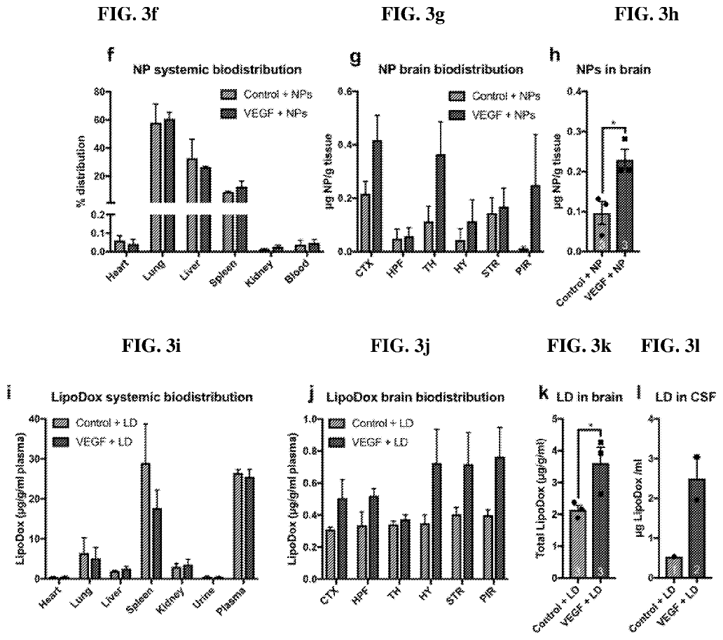

Lanyu mini pigs (n=3 per group) were administered VEGF (0.2 μg/kg) or a vehicle control via the carotid artery. Gd SNR enhancement was used to determine BBB integrity in multiple brain regions by MRI, as shown in FIG. 3a. As shown in FIG. 3b, pigs that received VEGF pre-treatment showed a significant increase in SNR as compared to those that received the vehicle control pre-treatment. Furthermore, the increase was quite consistent across multiple examined major brain ROIs. There was no significant difference in the SNR enhancement of the central cerebral sinus. The average SNR across all regions was four-fold higher (p=0.0035) in VEGF pre-treated pigs than the control vehicle pre-treated pigs. FIG. 3c. This level of increase is similar to the level of increase observed in mice (FIGS. 1c-1d). A heat map showing the change between normalised post vs pre signal intensity is also shown in the right panel of FIG. 3b.

A biodistribution study was also carried out in pigs using PEG-modified polystyrene nanoparticles (100 nm core diameter) and LipoDox as examples. FIG. 3d. A slight increase in total nanoparticle accumulation in the brain tissue of VEGF pre-treated pigs was observed. FIG. 3e. Precise HPLC-based quantification of systemic nanoparticle biodistribution 0 showed that the majority of the particles are accumulated in the lung. FIG. 3f. Comparison of specific brain regions showed an overall trend towards more nanoparticle retention after VEGF pre-treatment. FIG. 3g, left and right panels. Averaging all brain regions showed a small, but statistically significant increase (2.4-fold, p=0.0258) in nanoparticle retention in the brain. FIG. 3h.

Analysis of systemic LipoDox biodistribution by HPLC revealed a similar pattern to that observed in mice with most LipoDox remaining in circulation, and the spleen being the major organ of LipoDox retention. See FIG. 3i relative to FIG. 2c. Examination of region-specific LipoDox accumulation in the brain showed an overall trend towards more LipoDox in VEGF-pretreated animals. FIG. 3j. Averaging the whole brain data revealed a slight increase in LipoDox accumulation. FIG. 3k. Uncontaminated cerebrospinal fluid (CSF) was collected from three pigs. Two VEGF pre-treated animals both showed a higher LipoDox concentration in the CSF than the control treated animal. FIG. 3l.

Overall, these results demonstrate that VEGF-induced BBB permeability could be scaled up and induced in a large animal, which is more clinically relevant to human patients.

(iv) VEGF Affects Multiple Aspects of BBB Permeability

BBB permeability may be characterised by many changes including tight junction protein expression or altered localisation, pericyte detachment from endothelial cells, astrocyte loss, as well as changes in the activity of BBB transporters and efflux pumps. Mouse brains were collected 45 minutes or 4 hours following VEGF or saline injection and analysed for potential impact of VEGF on BBB permeability.

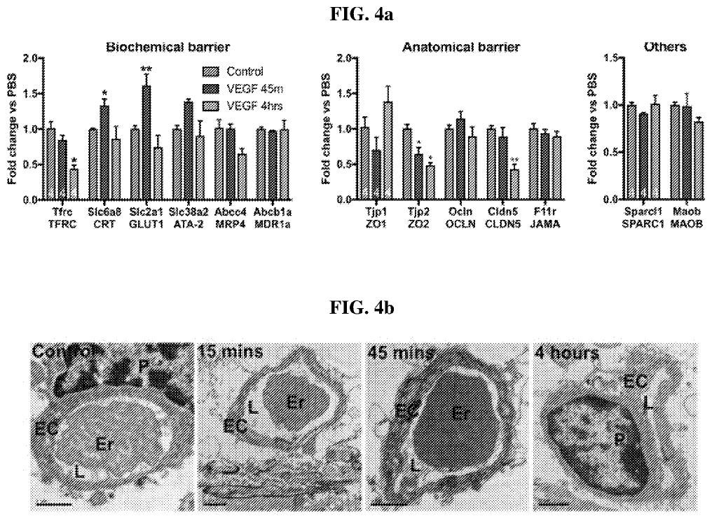

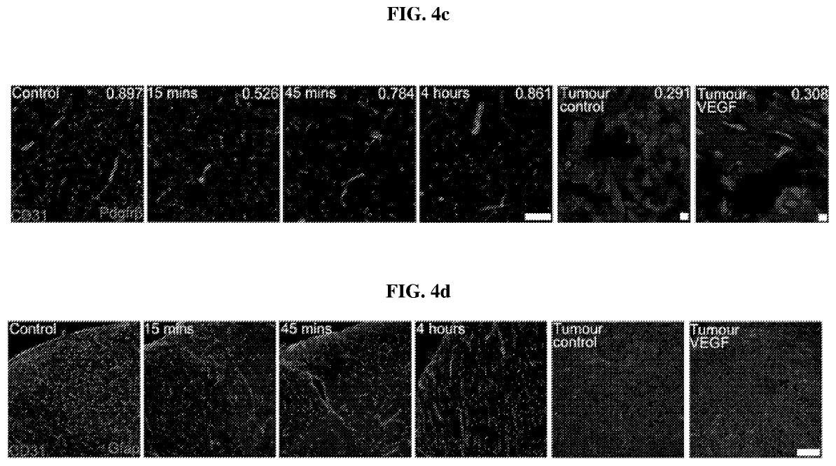

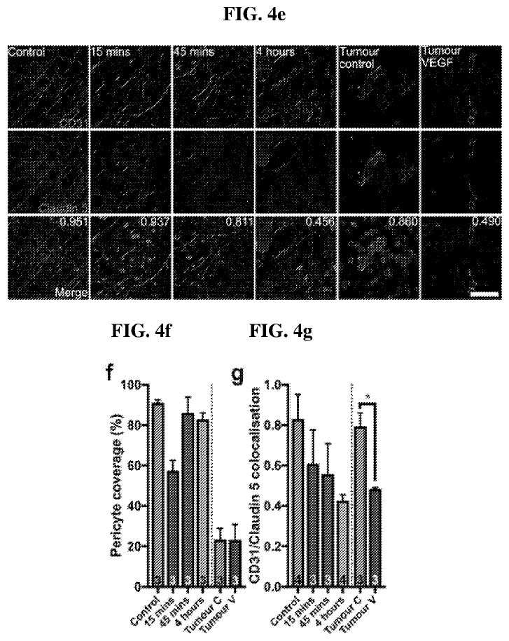

As an initial screen, the expression of key genes related to BBB integrity was measured. Macdonald et al., J. Neurosci. Methods, 174 (2), 219-226 (2008). Interestingly, the results show changes in the expression of several genes, including an increase in BBB transporters such as Slc2a1 (GLUT-1, glucose transporter) and Slc6a8 (CRT, creatinine transporter) and a decrease in tight junction components such as Tjp2 (ZO-2) and Cldn5 (Claudin 5), as shown in FIG. 4a.

Brains were taken from healthy mice at 45 minutes, 90 minutes, and 4 hours following VEGF injection, then frozen, sectioned, and stained for key indicators of BBB integrity. Transmission electron microscopy (TEM) was used to examine the morphology of brain blood vessels following VEGF treatment. Representative images show that in control animals, pericytes were present adjacent to endothelial cells, separated by a basement membrane, as normal. FIG. 4b. By contrast, at 15 minutes following VEGF injection, many vessels appeared slightly dilated and lacking adjacent pericytes. At 45 minutes, most vessels were no longer dilated, and at four hours following VEGF treatment, both vessels and pericytes appeared normal. FIG. 4b.

To further investigate this finding, frozen samples from GBM-bearing mice were taken at 45 minutes following VEGF injection. Pericytes were stained using antibodies against platelet-derived growth factor receptor beta (PDGFRβ), which has been previously used to visualise BBB integrity. Chang et al., Nat. Med., 23 (4) (2017). The length of pericyte coverage of blood vessels, stained with antibodies against CD31, was quantified, as shown in FIG. 4c. In control animals, PDGFRβ staining was observed outside of CD31⁺ blood vessels (91.1% coverage). However, at 15 minutes pericyte coverage was reduced (57.6%) and returned to normal after 45 minutes (86.2%) and 4 hours (83.0%), in line with observations from TEM. In the tumour region, blood vessels were highly variable in size and morphology, and showed little coverage with pericytes (30.8%). Surprisingly, pre-treatment with VEGF did not affect pericyte coverage (28.9%) within the tumour region.

GFAP, a marker of astrocytes, was also examined As shown in FIG. 4d, no obvious change in astrocyte morphology was apparent between treatment groups. Few astrocytes were present in the tumour region. Claudin 5, a component of endothelial cell tight junctions, was co-stained with the endothelial cell marker CD31. Ben-Zvi et al., Nature, 509 (7501), 507-511 (2014). The results show strong colocalisation (>95%) of claudin 5 and CD31 in control mice, which decreased at 45 minutes (55.8%) and 4 hours (42.7%) following VEGF administration, as shown in FIG. 4e. This result is in agreement with the gene expression data shown in FIG. 4a, which showed downregulation of Cldn5. In addition, a Western blot for Claudin-5 showed a trend towards reduced protein expression after VEGF administration (FIGS. 11a-11b). Interestingly, the tumour region still showed a large presence of tight junctions (80.0%) in control-treated mice. Following VEGF pre-treatment, this decreased to 48.5%. P-glycoprotein, the predominant efflux pump on the BBB, also appeared uniformly on the membrane of blood vessels at all time points, as shown in FIG. 11b. Kim et al., J. Clin. Invest., 126 (5), 1-17 (2016).

(v) VEGF in Combination with LipoDox Extends Survival in a Mouse Model of Glioblastoma

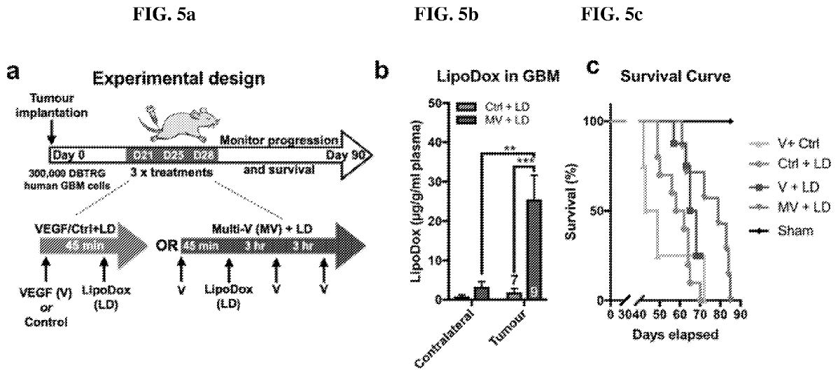

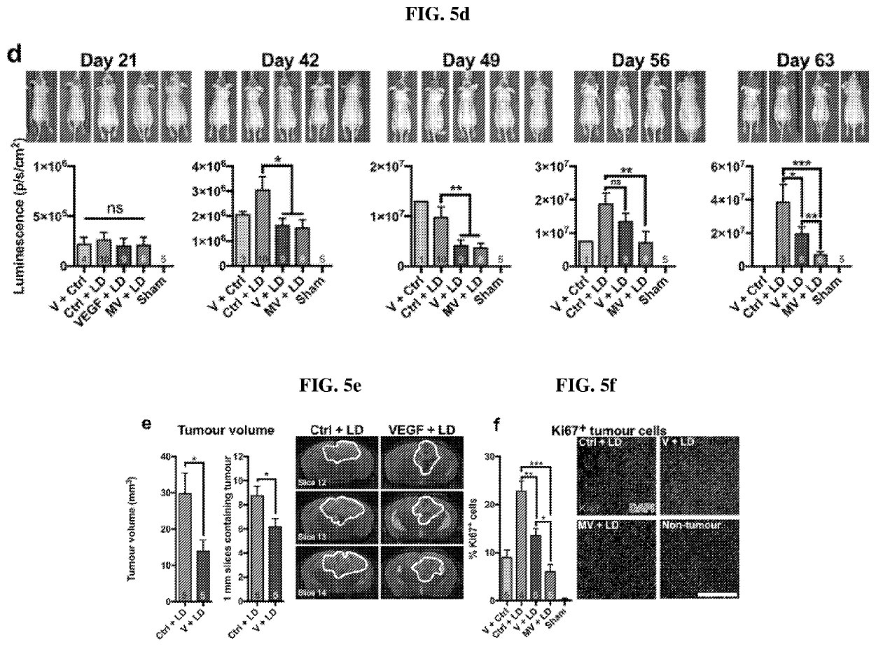

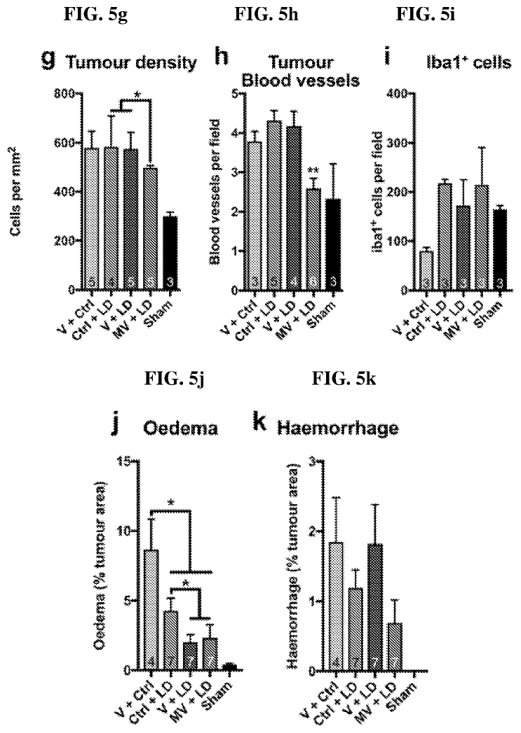

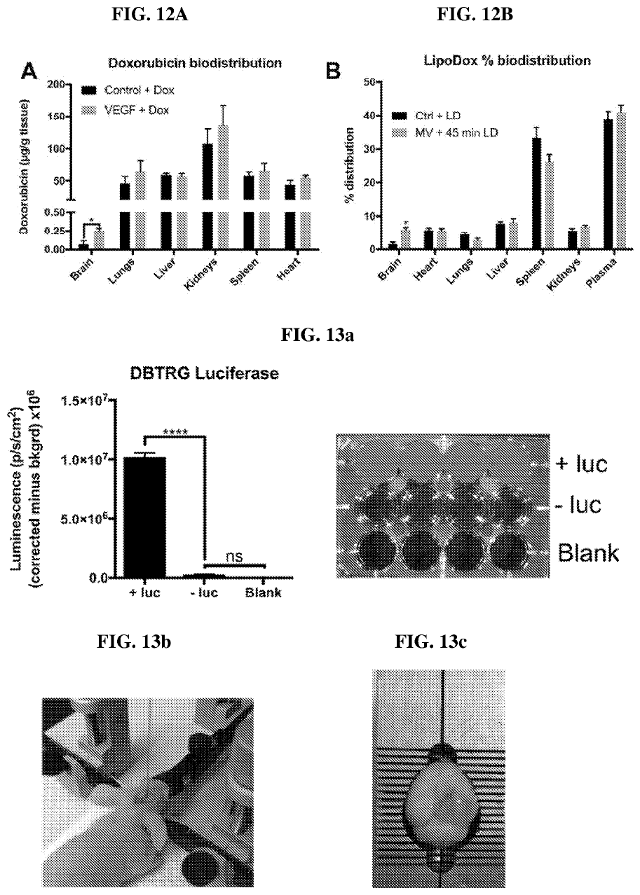

An experimental therapy of glioblastoma was carried out as outlined in FIG. 5a in a mouse glioblastoma model, using LipoDox in combination with VEGF pre-treatment. Given the long circulatory half-life of LipoDox (44.72 hours), and the transient nature of VEGF-induced BBB opening, it was expected that administration of multiple doses of VEGF (MV) after LipoDox administration (in addition to the VEGF pre-treatment) could provide multiple windows for brain delivery of LipoDox. MV mice were given VEGF first and then LipoDox at 45 minutes after the 1^{st} VEGF administration. The MV mice were further treated by two doses of VEGF at three hours and six hours after the LipoDox administration. Biodistribution of LipoDox in MV+VEGF mice is shown in FIG. 12B. As a comparison, biodistribution of doxorubicin in mice pre-treated with VEGF or a control was shown in FIG. 12A.

A human glioblastoma cell line, DBTRG-05MG, engineered to express luciferase, was used to form a xenograft glioblastoma model in BALB/c NU mice, as shown in FIG. 13a-c. See also FIG. 5a. Tumour progression was monitored by weekly IVIS and mice were assigned randomly to receive treatments of either VEGF+control (V+Ctrl), control+LipoDox (Ctrl+LD), VEGF+LipoDox (V+LD), or Multi-VEGF+LipoDox (MV+LD). Sham mice were intracranially injected with saline rather than tumour cells and received the MV+LD treatment course. LipoDox was given at a dose of 5 mg/kg, and treatments were given on Day 21, 25 and 28.

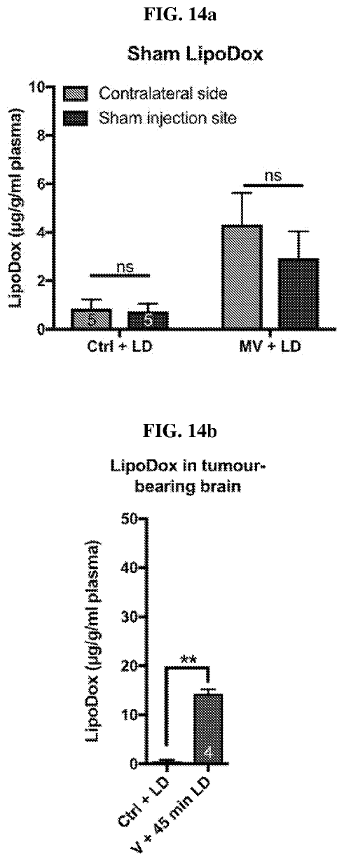

The delivery of LipoDox to GBM xenografts was quantified following VEGF pre-treatment. The results show that, surprisingly, intratumoural LipoDox was 7.8-fold higher than the contralateral side following VEGF pre-treatment. FIG. 5b. This was 13.6-fold more than the intratumoural concentration in control pre-treated mice. Importantly, the concentration detected in the tumour region of Ctrl+LD mice was only slightly higher (2.3-fold) than in the contralateral side, suggesting a small EPR effect from the tumour itself. It was also found that the sham injection procedure did not affect LipoDox accumulation (FIG. 14a), and a single injection of VEGF (V+LD) also increased intratumoural LipoDox, though to a lesser degree than MV+LD (FIG. 14b).

A Kaplan-Meier survival curve, shown in FIG. 5c, shows an improved median survival of V+LD (67 days, p=0.0271) and MV+LD (79 days, p=0.0042) groups compared to mice receiving Ctrl+LD (60 days). The difference between V+LD and MV+LD was also significant (p=0.0483). No sham-operated mice died during the course of the experiment.

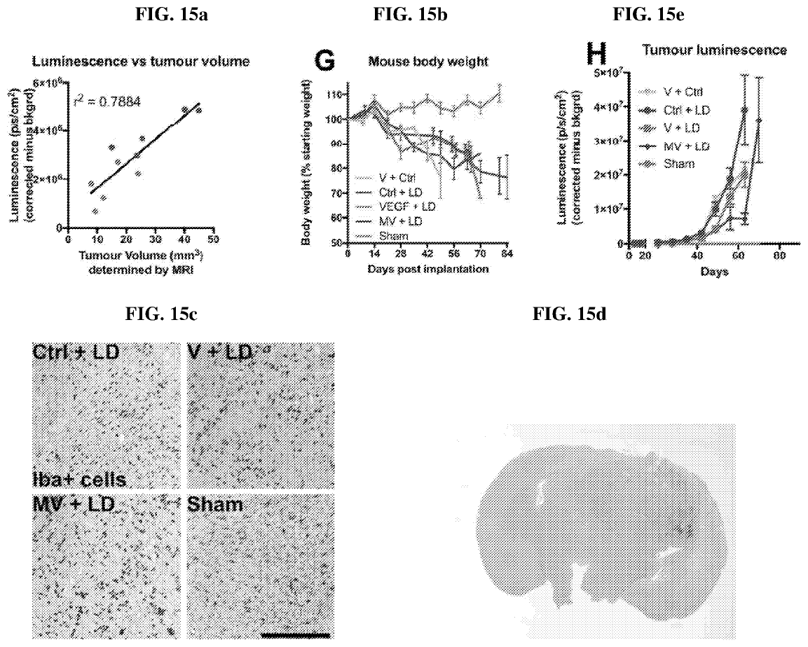

A weekly examination of tumour luminescence, shown in FIG. 5d, reveals no differences between the groups before the commencement of treatment (day 21). However, two weeks following completion of treatment (day 42), mice receiving V+LD and MV+LD treatments had significantly smaller tumours than Ctrl+LD mice (p=0.0425 and p=0.0417 respectively). This same trend continued at day 49 and day 56. By day 63 the MV+LD treated mice had significantly smaller tumours than the other groups (p=0.0029 vs. Ctrl+LD, p=0.01273 vs. V+LD). Representative IVIS images of mice from each group are shown above the corresponding graphs. Five mice each from the Ctrl+LD and V+LD treatment groups were randomly selected on day 45 to confirm tumour volume by MRI, as shown in FIG. 5e Image slices were captured and analysed by a blinded MRI technician. The results confirm that tumours in the V+LD treated mice were significantly smaller (p=0.0358) in total volume and were present in less MRI slices (p=0.0303) than Ctrl+LD treated mice, indicating delayed tumour progression. Representative image slices, with the tumour outlined, are shown. FIG. 15a shows that the correlation between IVIS luminescence and tumour volume confirmed by MRI is excellent (r-squared=0.7884). Mouse body weights are shown in FIG. 15b.

Samples from animals which died between days 60-70 were selected for staining V+Ctrl samples are included for reference, although these animals died before 60 days and so cannot be directly compared. FIG. 5f shows significant reductions in Ki67⁺ tumour cells in V+LD (p=0.0061) and MV+LD (p=0.0001) treated mice compared to Ctrl+LD treated mice, and between MV+LD and V+LD treated mice (p=0.0208). The overall cell density of the tumour determined by DAPI staining (FIG. 5g) was also slightly reduced in the MV+LD treated group (p=0.0478 vs. Ctrl+LD). V+Ctrl treated mice show less cell proliferation, likely due to the earlier time point of sample collection.

Given that VEGF is a potent stimulator of vasculogenesis, sections were stained with isolectin and blood vessels in the tumour were counted. In fact, tumours from mice in the MV+LD treatment groups showed less blood vessels than mice in the Ctrl +LD group (p=0.02) (FIG. 5h) Immunohistochemical staining for the microglial/macrophage marker Iba1 revealed no significant difference in the number of Iba1⁺ cells in the tumours of the various treatment groups, as shown in FIG. 5i. V+Ctrl treated mice showed less immune infiltration, again likely due to the earlier time point analysed. Example images of Iba1-stained tumours are shown in FIG. 15c.

Furthermore, since VEGF may increase interstitial fluid retention, H&E stained images were used to identify areas of oedema and haemorrhage within the tumour using ImageJ. An example H&E stained image is shown in FIG. 15d. Interestingly, there was a trend towards V/MV+LD treated animals showing less oedema than control treated animals (FIG. 5j). There was no significant difference in haemorrhage between groups, although it was highly variable between individual animals (FIG. 5k).

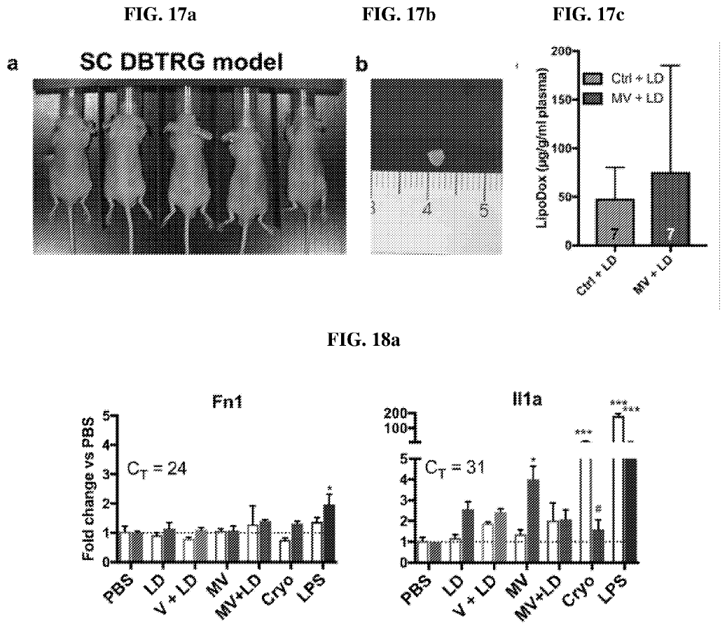

To examine whether VEGF may act on other malignant tumours, LipoDox uptake was quantified in pancreatic ductal adenocarcinoma (PDAC) model (FIG. 16a-16c) following the Ctrl/MV+LD protocol, and compared uptake by the normal pancreas and the tumour xenograft. The results (FIG. 16d) show that PDAC xenografts took up ˜3-fold more LipoDox than the sham-operated pancreas. This suggests some EPR effect is present in this model, although the overall concentration of LipoDox is still low. The addition of VEGF pre-treatment did not change uptake in either the normal pancreas or the PDAC xenograft. This is in agreement with previous data showing that VEGF at this dose does not cause changes in vascular permeability of peripheral organs. The effect of VEGF pre-treatment on LipoDox accumulation was examined in GBM xenografts placed subcutaneously. As shown in FIGS. 17a-17c, no significant change in LipoDox accumulation was found in the tumour following the MV+LD treatment protocol. Of note, the LipoDox concentration in control-treated tumours was 25.8× higher in subcutaneous vs orthotopic xenografts, since the tumours are no longer sheltered by the BBB.

(vi) Low Dose Intravenous Administration of VEGF is Safe

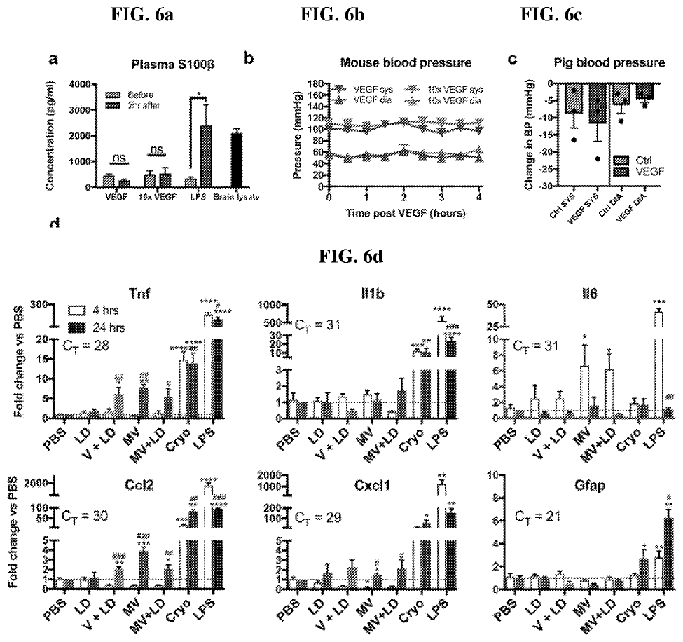

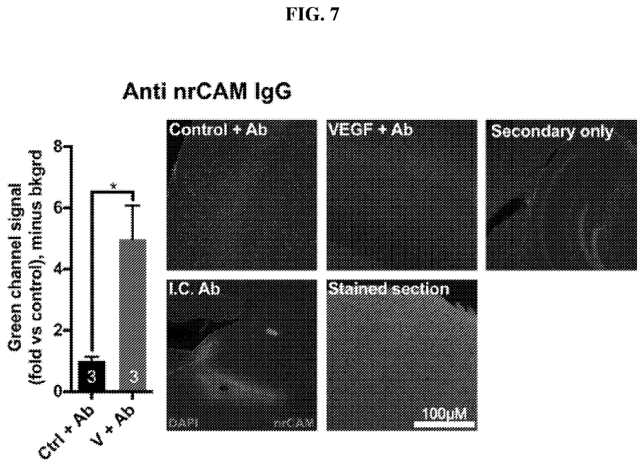

Administration of VEGF was suggested to disrupt compartmentalisation of the brain, resulting in the escape of brain components into systemic circulation. This potential side effect of using VEGF to facilitate delivery of therapeutic agents to brain is examined The calcium-binding protein S100 beta (S100β) was used as a marker in this study. The presence of this protein in blood has been previously shown to serve as a peripheral marker of brain injury and loss of BBB integrity. Marchi et al., Clin. Chim. Acta., 342 (1-2), 1-12 (2004); and Plog et al., J Neurosci, 35 (2), 518-526 (2015). The results, shown in FIG. 6a, show no significant change in mouse plasma S100β two hours following administration of VEGF at the low dose disclosed herein, or at a 10-fold higher dose of VEGF. Lipopolysaccharide (LPS), a potent inducer of neuroinflammation which increases BBB permeability, was used as a positive control and caused a significant elevation of plasma S100β two hours following administration.

In human clinical trials, VEGF was found to induce temporary systemic hypotension during infusion. Henry et al., Circulation, 107 (10), 1359-1365 (2003); and Eppler et al., Clin. Pharmacol. Ther., 72 (1), 20-32 (2002). To investigate whether the given bolus dose could cause the same effect, mice were injected with VEGF at the low dose, or a ten-fold higher dose, and blood pressure was measured every 30 minutes using a BP-2000 Series II Blood Pressure Analysis System. The results, shown in FIG. 6b, show no notable change in blood pressure over a four-hour period following VEGF administration. Similarly, no clear changes in blood pressure were seen in the pigs which received VEGF compared to control (FIG. 6c), although an overall trend towards decreased blood pressure was seen in both groups, likely due to anaesthesia and surgery. Interestingly, a rapid, but temporary, flushing reaction in one pig which received VEGF was observed—a phenomenon which has also been observed in humans Henry et al., Am. Heart J., 142 (5), 872-880 (2001).

Endogenous VEGF is known to induce neuroinflammation following brain injury. Argaw et al., J. Clin. Invest. 2012,122 (7), 2454-2468 (2012). However, the effects of exogenous intravenous VEGF on the brain are unclear, given that many VEGF receptors are present on the abluminal, brain-facing side of brain endothelial cells. Kaya et al., J. Cereb. Blood Flow Metab., 25 (9), 1111-1118 (2005). Therefore, to gain insight into whether intravenous VEGF may also induce neuroinflammation, real time quantitative PCR was used to screen for changes in the gene expression of several major cytokines related to neuroinflammation. Skelly et al., PLoS One, 8 (7), 1-20 (2013); and Monnet-Tschudi et al., Curr. Protoc. Toxicol., No. SUPPL. 50, 1-20 (2011). Animals were perfused at four hours or 24 hours following administration of the VEGF and LipoDox treatment groups used in this study. Cryolesion and LPS were both used as positive controls. The results, shown in FIG. 6d, indicate that VEGF administration moderately increased the expression of a number of neuroinflammation-related genes. Expression of Tnfa, Ccl2 and Cxcl1 was found to be unchanged four hours after VEGF treatment, but was moderately increased 24 hours after the treatment. The gene expression of the acute inflammation marker Il6 was increased after 4 hours in treatment groups utilising multi-VEGF, but not single VEGF. No treatment group significantly increased Il1b or Gfap expression, although both were raised by cryolesion or LPS.

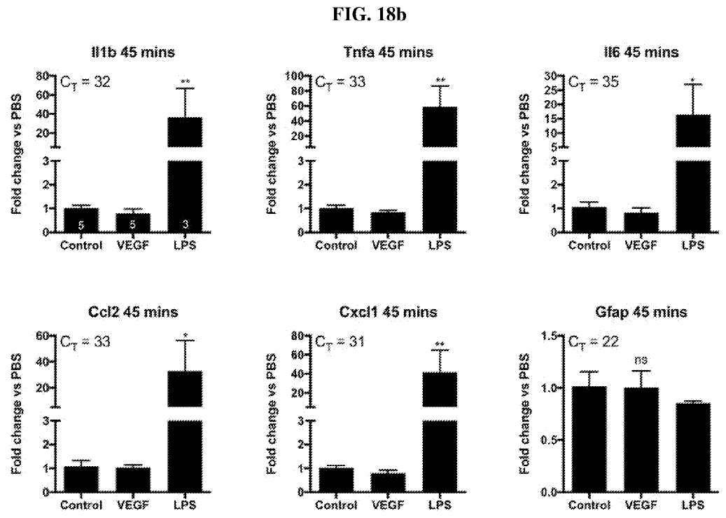

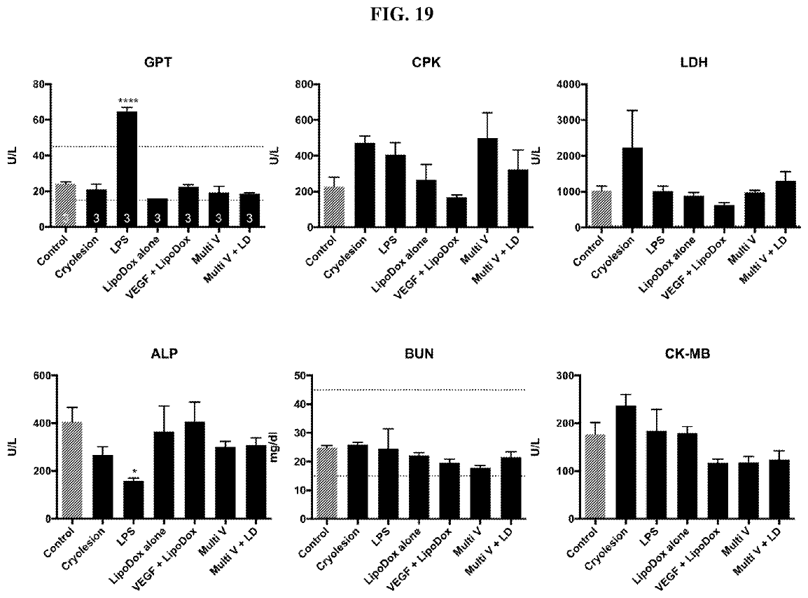

Gene expression data for additional inflammation markers is shown in FIG. 18a, and a list of all primers used is in shown in Table 1 above. Measurements taken at the 45 minutes following VEGF administration show no elevation of these same genes compared to controls, indicating that inflammation may be a delayed response—potentially a response to enhanced BBB permeability. FIG. 18b. In addition, blood chemistry results for liver and kidney function showed no adverse changes following treatment. FIG. 19. These results demonstrate that the given dose of VEGF appears safe.

### Discussions

It is interesting that VEGF is specifically effective in enhancing BBB penetration of molecules such as 20 nm-100 nm nanoparticles, and LipoDox (˜95 nm diameter), all readily passed into the brain following VEGF pre-treatment. LipoDox is currently used for treatment of solid tumours in the breast and ovary but has not approved for treating GBM. LipoDox may be more effective than doxorubicin in patients whose tumours express p-glycoprotein, since PEG modification shields the drug molecule from efflux, and may allow it to pass more easily within the brain tissue. Nance et al., Sci Transl Med, 4 (149), 149ra119 (2012).

MRI analysis based on gadolinium contrast enhancement showed very similar results in pigs (˜4-fold increase in SNR) to those observed in mice. This is encouraging, given that the MRI is measuring the real-time signal in the living brain, whereas other methods rely on post-mortem collection of tissues, drug extraction and quantification. MRI also allows for before-after comparisons from the same animal, countering inherent heterogeneity between animals.

The results show herein decreased gene expression of Tjp2 (ZO-2) and Cldn5 in the brain soon after VEGF administration. Staining of brain sections following VEGF also confirmed these findings. The tumour model is slow-growing (median survival 50-60 days without treatment) and still showed a high degree of tight junction colocalization with endothelial cells, indicating that the BBTB is relatively intact. Indeed, it was found that only 2.3-fold more LipoDox entered the tumour compared to the contralateral healthy side. When the same tumour cell line was used to establish subcutaneous GBM xenografts, the LipoDox concentration in the tumour was 25 times higher than for orthotopic xenografts, clearly demonstrating how the BBB prevents effective drug delivery to the brain.

Endogenous VEGF is known to modulate astrocyte activation, which in turn mediates BBB integrity. This is particularly relevant during the response to injury such as ischaemia, where astrocyte-secreted VEGF locally increases BBB permeability. Argaw et al., 2012. However, no change in astrocyte morphology or Gfap gene expression under the conditions analysed was observed. Previous studies have found that exogenous VEGF can modulate p-glycoprotein activity in isolated brain capillaries and in situ rat brains. Hawkins et al., J. Neurosci. 2010, 30 (4), 1417-1425 (2010). No change was found in p-glycoprotein gene expression (Abcb1a), or the morphological appearance of p-glycoprotein staining in frozen sections following VEGF administration at the given dose (FIG. 11b). However, in tumour xenografts, pericyte coverage of blood vessels was already significantly reduced compared to healthy brain, and was not further reduced by VEGF pre-treatment. Thus, it is speculated that intravenous VEGF increases BBB permeability through transient degradation of brain endothelial cell tight junctions, although other mechanisms may also be involved.

In terms of safety, it was found that intravenous VEGF increased the expression of a number of neuroinflammation-related genes in the brains in otherwise healthy mice. Neuroinflammation is a complex multi-faceted process involving local production of cytokines as well as increased activity of BBB cytokine transporters which allow more externally produced cytokines into the brain. Obermeier et al., Nat Med, 19 (12), 1584-1596 (2013).

In summary, the results reported herein have shown that a low dose of intravenous VEGF improved the delivery of nanomedicine therapeutics to the brain. The findings have potential for translation to the clinic in order to enable improved therapy of brain tumours, which is an unmet clinical need of upmost urgency.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method for delivering a therapeutic agent to the brain of a subject, the method comprising:
(i) administering a first dose of a vascular endothelial growth factor (VEGF) polypeptide systemically to a subject in need thereof;
(ii) administering to the subject systemically an effective amount of a therapeutic agent 15 minutes to 3 hours after step (i); and
(iii) administering systemically a second dose of the VEGF polypeptide to the subject 2-24 hours after step (ii).

2. The method of claim 1, wherein the second dose of the VEGF polypeptide in step (iii) is administered 2-8 hours after administration of the therapeutic agent in step (ii), optionally 3-5 hours after administration of the therapeutic agent in step (ii).

3. The method of claim 1 or claim 2, wherein the method further comprises (iv) administering a third dose of the VEGF polypeptide 2-24 hours after the second dose of the VEGF polypeptide in step (iii), preferably 2-12 hours after the second dose of the VEGF polypeptide in step (iii), and more preferably 3-5 hours after the second dose of the VEGF polypeptide in step (iii).

4. The method of any one of claims 1-3, wherein the therapeutic agent is administered to the subject about 45 minutes after the first dose of the VEGF polypeptide in step (i).

5. The method of any one of claims 1-4, wherein the second dose of the VEGF polypeptide in step (iii) is administered to the subject about 3 hours after administration of the therapeutic agent in step (ii).

6. The method of any one of claims 3-5, wherein the third dose of the VEGF polypeptide in step (iv) is administered to the subject about 3 hours after administration of the second dose of the VEGF polypeptide in step (iii).

7. The method of any one of claims 1-6, wherein the first dose, the second dose, and/or the third dose of the VEGF polypeptide is about 50-200 ng/kg.

8. The method of claim 7, wherein the first dose, the second dose, and/or the third dose of the VEGF polypeptide is about 100-150 ng/kg.

9. The method of any one of claims 1-8, wherein the VEGF polypeptide is a VEGF-A polypeptide, optionally wherein the VEGF-A polypeptide is human VEGF165A.

10. The method of any one of claims 1-9, wherein the therapeutic agent is encapsulated by or attached to a liposome or a nanoparticle.

11. The method of claim 10, wherein the liposome or the nanoparticle is pegylated.

12. The method of claim 10 or claim 11, wherein the liposome or the nanoparticle has a solid core diameter of about 20-500 nm, optionally about 20-300 nm.

13. The method of any one of claims 1-12, wherein the therapeutic agent is formulated in a pharmaceutical composition, which further comprises a pharmaceutically acceptable carrier.

14. The method of any one of claims 1-12, wherein the therapeutic agent is in free form. The method of any one of claims 1-4, wherein the therapeutic agent is a small molecule, a protein, or a nucleic acid.

16. The method of any one of claims 1-15, wherein the therapeutic agent is water soluble and has a molecular weight greater than 500 Dalton, optionally wherein the therapeutic agent is doxorubicin.

17. The method of any one of claims 1-16, wherein the subject is a human patient suspected of having, is at risk for, or a brain disease, which optionally is selected from the group consisting of a brain tumor, a brain stroke, a neuropsychiatric disorder, and a neurodegenerative disease.

18. The method of any one of claims 1-17, wherein the first dose of VEGF and/or the second dose of VEGF is administered via an intravenous injection or intra-arterial injection.

19. A method for delivering a therapeutic agent to the brain of a subject, the method comprising:
(i) administering a vascular endothelial growth factor (VEGF) polypeptide systemically to a subject in need thereof at a dose of about 50-200 ng/kg;
(ii) administering to the subject a therapeutic agent 15 minutes to 3 hours after step (i).

20. The method of claim 19, wherein the therapeutic agent is administered to the subject about 45 minutes after step (i).

21. The method of claim 19 or claim 20, wherein the dose of the VEGF polypeptide in step (i) is about 100-150 ng/kg.

22. The method of any one of claims 19-21, wherein the therapeutic agent is encapsulated by or attached to a liposome.

23. The method of claim 22, wherein the therapeutic agent is doxorubicin.

24. The method of any one of claims 19-23, wherein the subject is a human patient suspected of having, is at risk for, or a brain disease, which optionally is selected from the group consisting of a brain tumor, a brain stroke, a neuropsychiatric disorder, and a neurodegenerative disease.

25. The method of any one of claims 19-24, wherein the VEGF is administered via an intravenous injection or intra-arterial injection.


SEQUENCE LISTING (45 sequences)

SEQ ID NO: 1 - protein, 165 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 2 - DNA, 20 bp, Mus musculus
acccagaagactgtggatgg

SEQ ID NO: 3 - DNA, 20 bp, Mus musculus
cacattgggggtaggaacac

SEQ ID NO: 4 - DNA, 22 bp, Mus musculus
gaccctcacactcagatcttct

SEQ ID NO: 5 - DNA, 17 bp, Mus musculus
cctccacttggtttgct

SEQ ID NO: 6 - DNA, 21 bp, Mus musculus
tgccaccttttgacagtgatg

SEQ ID NO: 7 - DNA, 14 bp, Mus musculus
atgtgcgagatttg

SEQ ID NO: 8 - DNA, 22 bp, Mus musculus
tccagaaaccgctatgaagttc

SEQ ID NO: 9 - DNA, 20 bp, Mus musculus
caccagcatcagtcccaaga

SEQ ID NO: 10 - DNA, 19 bp, Mus musculus
gttggctcagccagatgca

SEQ ID NO: 11 - DNA, 21 bp, Mus musculus
agcctactcattgggatcttg

SEQ ID NO: 12 - DNA, 21 bp, Mus musculus
gaacaacctggctgcgtatag

SEQ ID NO: 13 - DNA, 22 bp, Mus musculus
gcgattcaacctttctctccaa

SEQ ID NO: 14 - DNA, 21 bp, Mus musculus
cacccaaaccgaagtcatagc

SEQ ID NO: 15 - DNA, 23 bp, Mus musculus
aattttctgaaccaagggagctt

SEQ ID NO: 16 - DNA, 18 bp, Mus musculus
aggcaatggacgcatcac

SEQ ID NO: 17 - DNA, 17 bp, Mus musculus
ctcggttgtccttcttg

SEQ ID NO: 18 - DNA, 22 bp, Mus musculus
cgcttgagtcggcaaagaaatc

SEQ ID NO: 19 - DNA, 22 bp, Mus musculus
cagagagagatggtcaatggca

SEQ ID NO: 20 - DNA, 22 bp, Mus musculus
ctgctcatcactatggtggcta

SEQ ID NO: 21 - DNA, 20 bp, Mus musculus
tgaccccatggcaaaactga

SEQ ID NO: 22 - DNA, 22 bp, Mus musculus
gtgggggtaagggtggaatgta

SEQ ID NO: 23 - DNA, 21 bp, Mus musculus
gccacaactacacactcccaa

SEQ ID NO: 24 - DNA, 20 bp, Mus musculus
tggcgggagacgcatagtta

SEQ ID NO: 25 - DNA, 15 bp, Mus musculus
gcccgtcaccttgct

SEQ ID NO: 26 - DNA, 24 bp, Mus musculus
acgaaacagactttcatccaggta

SEQ ID NO: 27 - DNA, 20 bp, Mus musculus
aagcccaaggattccactgc

SEQ ID NO: 28 - DNA, 16 bp, Mus musculus
gggcgagatgctgccg

SEQ ID NO: 29 - DNA, 20 bp, Mus musculus
gggttgagccaccagaagaa

SEQ ID NO: 30 - DNA, 20 bp, Mus musculus
ccatcttccaaggctctgct

SEQ ID NO: 31 - DNA, 20 bp, Mus musculus
ccatcacgacctcacgtgtc

SEQ ID NO: 32 - DNA, 20 bp, Mus musculus
cctgtgaagcgtcactgtgt

SEQ ID NO: 33 - DNA, 20 bp, Mus musculus
cgcggagagagacaagatgt

SEQ ID NO: 34 - DNA, 16 bp, Mus musculus
gagatgccggtgcggg

SEQ ID NO: 35 - DNA, 20 bp, Mus musculus
tttggaatccttctgcaggg

SEQ ID NO: 36 - DNA, 20 bp, Mus musculus
catagtcagatgggggtgga

SEQ ID NO: 37 - DNA, 20 bp, Mus musculus
atttatgatgaacagccccc

SEQ ID NO: 38 - DNA, 20 bp, Mus musculus
gtcacgatgttgtggtccag

SEQ ID NO: 39 - DNA, 20 bp, Mus musculus
aaattctgggtctggtgctg

SEQ ID NO: 40 - DNA, 20 bp, Mus musculus
agtgtacaccgaacccttgc

SEQ ID NO: 41 - DNA, 20 bp, Mus musculus
tgtaactgtaatgggcaccg

SEQ ID NO: 42 - DNA, 21 bp, Mus musculus
acctctccgcagatctagcca

SEQ ID NO: 43 - DNA, 20 bp, Mus musculus
ggtgtcaccagtgttgcagt

SEQ ID NO: 44 - DNA, 22 bp, Mus musculus
gctggaccaaatctacaaagca

SEQ ID NO: 45 - DNA, 20 bp, Mus musculus
tggttgtacctccacactgc